(19) United States
(12) Patent Application Publication
(10) Pub. No.: US 2024/0226028 A1
(43) Pub. Date: Jul. 11, 2024

(54) LYOPHILIZATION METHODS FOR PREPARING LIPID FORMULATED THERAPEUTICS

(71) Applicant: ModernaTX, Inc., Cambridge, MA (US)

(72) Inventors: Johnathan Goldman, Cambridge, MA (US); Kimberly Hassett, Cambridge, MA (US); Xiaohan Peng, Norwood, MA (US); Sarah Sullivan, Cambridge, MA (US)

(73) Assignee: ModernaTX, Inc., Cambridge, MA (US)

(21) Appl. No.: 18/288,328

(22) PCT Filed: Apr. 29, 2022
(86) PCT No.: PCT/US2022/027043

§ 371 Date: Oct. 25, 2023

Related U.S. Application Data
(60) Provisional application No. 63/210,389, filed on Jun. 14, 2021.
(60) Provisional application No. 63/181,592, filed on Apr. 29, 2021.

(51) Int. Cl. A61K 9/51; A61K 9/19

(52) U.S. Cl. CPC A61K 9/5192; A61K 9/19; A61K 9/5123

(57) ABSTRACT

Aspects of the present disclosure relate to methods of preparing lyophilized compositions comprising lipid, nanoparticles and nucleic acids. Other aspects relate to lyophilized compositions with low moisture contents and improved stability during long-term storage.

### RELATED APPLICATIONS

This application claims the benefit under 35 U.S.C. § 119(e) of U.S. provisional Application No. 63/181,592 filed Apr. 29, 2021, and U.S. provisional Application No. 63/210,389 filed Jun. 14, 2021, each of which is incorporated by reference herein in its entirety.

### BACKGROUND

Delivery of mRNA formulated in lipid nanoparticles (LNPs) and subsequent cellular uptake of the mRNA allows desired proteins, such as viral or bacterial antigens, to be expressed in vivo. However, LNPs, and the mRNA inside them, can be degraded by environmental conditions, such as the presence of water or environmental nucleases. This instability raises challenges for extended storage of LNP-mRNA compositions.

### SUMMARY

Aspects of the disclosure relate to methods of lyophilizing lipid nanoparticles (LNPs) comprising nucleic acids, such as mRNA, to produce lyophilized compositions with low moisture contents. Lyophilization, or freeze-drying, is a method of preserving a composition by freezing the composition, incubating the frozen composition at a low temperature and pressure to remove water by sublimation (primary drying phase), and finally incubating the composition at a higher temperature to remove residual water that is bound to the composition (secondary drying phase). Remaining water in a lyophilized composition can facilitate the degradation of nucleic acids via hydrolysis, and thus minimizing the moisture content of a lyophilized composition is important for producing lyophilized compositions in which nucleic acids are stable during long-term storage. The final moisture content of a lyophilized composition can be reduced by heating the composition to a higher temperature during the secondary drying phase, but doing so reduces the integrity of the composition. Unexpectedly, the introduction of an annealing step during the freezing phase, in which lipid nanoparticles were held at a temperature near the freezing point of water prior to deep freezing, allowed for the use of a higher temperature during the secondary drying phase without compromising nucleic acid integrity. Furthermore, the inclusion of sucrose as a lyoprotectant unexpectedly resulted in a reduction in LNP size, increasing the efficiency of the LNP-mRNA production process. These features resulted in the preparation of lyophilized compositions of mRNA formulated in LNPs with lower moisture contents than were previously possible. The mRNA in the resulting lyophilized compositions thus decays more slowly, allowing these compositions to remain stable during extended storage, while still retaining the ability to be translated into an encoded protein following reconstitution of the composition and introduction of the mRNA into cells.

Accordingly, some aspects of the disclosure relate to a method of preparing a lyophilized composition, the method comprising lyophilizing a lipid nanoparticle composition that comprises a lipid nanoparticle and an mRNA.

In some embodiments, the lipid nanoparticle composition comprises a lyoprotectant.

In some embodiments, the lyoprotectant comprises a sugar.

In some embodiments, the lyoprotectant comprises sucrose.

In some embodiments, the lipid nanoparticle comprises a lipid, and the lipid nanoparticle composition comprises a lyoprotectant mass:lipid mass ratio of at least 5:1.

In some embodiments, the lyoprotectant mass:lipid mass ratio is about 6:1 to about 40:1, optionally wherein the lyoprotectant mass lipid mass ratio is about 6:1, 7:1, 8:1, 9:1, 10:1, 15:1, 20:1, 20:1, 30:1, or 40:1.

In some embodiments, the composition comprises a buffer.

In some embodiments, the buffer is selected from the group consisting of a Tris buffer, citrate buffer, and phosphate buffer.

In some embodiments, the buffer is a Tris buffer.

In some embodiments, the concentration of the buffer in the composition is about 1 mM to about 100 mM.

In some embodiments, the concentration of the buffer is about 10 mM to about 20 mM.

In some embodiments, the composition has a pH of about 6.5 to about 8.5.

In some embodiments, the composition has a pH of about 7 to about 8.

In some embodiments, the composition has a pH of about 7.4 to about 8.

In some embodiments, the lyophilizing comprises an annealing step. In some embodiments, the annealing step comprises exposing the lipid nanoparticle composition to a first temperature above the freezing temperature of the composition and a second temperature below the freezing temperature of the composition.

In some embodiments, the first temperature is from about −30° C. to about 0° C.

In some embodiments, the first temperature is about −30° C., about −25° C., about −20° C., about −15° C., about −10° C., about −5° C., or about 0° C.

In some embodiments, the second temperature is from about −100° C. to about −30° C.

In some embodiments, the second temperature is about −100° C., about −90° C., about −80° C., about −70° C., about −60° C., about −50° C., about −40° C., or about −30° C.

In some embodiments, the method comprises exposing the lipid nanoparticle composition to a third temperature before exposing the lipid nanoparticle composition to the first temperature, wherein the third temperature is from about −100° C. to about −30° C.

In some embodiments, the third temperature is about −100° C., about −90° C., about −80° C., about −70° C., about −60° C., about −50° C., about −40° C., or about −30° C.

In some embodiments, the annealing step is conducted for at least 2 hours, at least 3 hours, at least 4 hours, at least 5 hours, at least 10 hours, or at least 15 hours.

In some embodiments, the lipid nanoparticle composition is exposed to the first temperature for a first period of from about 2 to about 6 hours, the second temperature for a second period of from about 2 to about 6 hours, and/or the third temperature for a third period of from about 2 to about 6 hours.

In some embodiments, the lyophilizing comprises a sublimation step.

In some embodiments, the sublimation is performed at a vacuum pressure of from about 50 mTorr and about 300 mTorr.

In some embodiments, the lyophilizing comprises a desorption step, wherein the desorption step comprises exposing the composition to a desorption temperature.

In some embodiments, the desorption temperature is about 10° C. or higher.

In some embodiments, the desorption temperature is about 10° C., about 15° C., about 20° C., about 25° C., about 30° C., about 35° C., about 40° C., about 45° C., about 50° C., about 55° C., or about 60° C.

In some embodiments, the desorption step comprises exposing a sublimated composition to a desorption temperature of about 40° C.

In some embodiments, the desorption is conducted for at least 2 hours.

In some embodiments, the desorption is conducted for about 5 hours, about 10 hours, about 15 hours, or about 20 hours.

In some embodiments, the desorption is conducted until a Pirani gauge measuring a relative vacuum at the desorption temperature produces a reading that is about the same as the reading produced by a capacitance manometer measuring absolute pressure at the desorption temperature.

In some embodiments, the desorption is performed at a vacuum pressure of from about 50 mTorr and about 300 mTorr.

In some embodiments, the lyophilized composition has a moisture content of 6.0% w/w or less 5.0% w/w or less, 4.0% w/w or less, 3.5% w/w or less, 3.0% w/w or less, 2.5% w/w or less, 2.0% w/w or less, 1% or less, 0.5% or less, 0.3% or less, or 0.25% or less.

In some embodiments, a coefficient of degradation at 5° C. of the nucleic acid in the lyophilized composition is 0.05 month⁻¹ or less, 0.04 month⁻¹ or less, 0.03 month⁻¹ or less, or 0.02 month⁻¹ or less.

In some embodiments, the coefficient of degradation is 0.01 month⁻¹ or less, 0.008 month⁻¹ or less, 0.006 month⁻¹ or less, or 0.004 month⁻¹ or less.

In some embodiments, the mRNA in the lyophilized composition is at least 50% pure after least 12 months, at least 18 months, at least 21 months, at least 24 months, at least 27 months, at least 30 months, at least 33 months, or at least 36 months of storage.

In some embodiments, the storage is conducted at a temperature between about 2° C. and about 8° C.

In some embodiments, the storage is conducted at about 5° C.

In some embodiments, the mRNA in the lyophilized composition is at least 50% pure after at least 48 months or at least 60 months of storage at about 5° C.

In some embodiments, a high performance liquid chromatography analysis of the mRNA in the lyophilized composition produces a chromatogram comprising N peaks, wherein one of the N peaks is a main peak, wherein the main peak measures mRNA that was present in the composition before lyophilization, wherein the area under the curve (AUC) of each peak is calculated by integration, wherein the purity of the mRNA in the lyophilized composition is calculated using the equation

$$\% purity = \frac{A U C ( main peak )}{{\sum }_{i = 1}^{N} A U C ( {peak}_{i} )} .$$

In some embodiments, the lipid nanoparticle comprises: an ionizable amino lipid.

In some embodiments, the lipid nanoparticle further comprises: a non-cationic lipid; a sterol; and a polyethylene glycol (PEG)-modified lipid.

In some embodiments, the lipid nanoparticle comprises: 40-55 mol % ionizable amino lipid; 5-15 mol % non-cationic lipid; 35-45 mol % sterol; and 1-5 mol % PEG-modified lipid.

Some aspects of the disclosure relate to a lyophilized composition produced by a method of any one of the methods described herein.

Some aspects of the disclosure relate to a lyophilized pharmaceutical composition comprising a lipid nanoparticle and an mRNA.

In some embodiments, the lyophilized pharmaceutical composition comprises a lyoprotectant.

In some embodiments, the lyoprotectant comprises a sugar.

In some embodiments, the lyoprotectant comprises sucrose.

In some embodiments, the lipid nanoparticle comprises a lipid, and where the lyophilized pharmaceutical composition comprises a lyoprotectant mass:lipid mass ratio of at least 5:1.

In some embodiments, the lyoprotectant mass:lipid mass ratio is about 6:1 to about 40:1, optionally wherein the lyoprotectant mass lipid mass ratio is about 6:1, 7:1, 8:1, 9.1, 10:1, 15:1, 20:1, 20:1, 30:1, or 40:1.

In some embodiments, the lyophilized composition has a moisture content of 6.0% w/w or less, 5.0% w/w or less, 4.0% w/w or less, 3.5% w/w or less, 3.0% w/w or less, 2.5% w/w or less, or 2.0% w/w or less, 1% or less, 0.5% or less, 0.3% or less, or 0.25% or less.

In some embodiments, a coefficient of degradation at 5° C. of the nucleic acid in the lyophilized composition is 0.05 month⁻¹ or less, 0.04 month⁻¹ or less, 0.03 month⁻¹ or less, or 0.02 month⁻¹ or less.

In some embodiments, the coefficient of degradation is 0.01 month⁻¹ or less, 0.008 month⁻¹ or less, 0.006 month⁻¹ or less, or 0.004 month⁻¹ or less.

In some embodiments, the mRNA in the lyophilized composition is at least 50% pure after least 12 months, at least 18 months, at least 21 months, at least 24 months, at least 27 months, at least 30 months, at least 33 months, or at least 36 months of storage.

In some embodiments, the storage is conducted at a temperature between about 2° C. and about 8° C.

In some embodiments, the storage is conducted at about 5° C.

In some embodiments, the mRNA in the lyophilized composition is at least 50% pure after at least 48 months or at least 60 months of storage at about 5° C.

In some embodiments, the lipid nanoparticle comprises: an ionizable amino lipid.

In some embodiments, the lipid nanoparticle further comprises: a non-cationic lipid; a sterol; and a polyethylene glycol (PEG)-modified lipid.

In some embodiments, the lipid nanoparticle comprises: 40-55 mol % ionizable amino lipid; 5-15 mol % non-cationic lipid; 35-45 mol % sterol; and 1-5 mol % PEG-modified lipid.

Some aspects of the disclosure relate to a method comprising reconstituting any one of the lyophilized pharmaceutical compositions described herein.

### BRIEF DESCRIPTION OF THE DRAWINGS

FIG. 1 shows the delta diameter of lipid nanoparticles comprising mRNA, which varies based on the concentration of mRNA to be encapsulated, the sucrose:lipid ratio, and the liquid in which the nanoparticles are reconstituted.

FIGS. 2A-2C show the parameters of two example lyophilization processes, one with an annealing step and one without, and the effects of each process on the characteristics of lipid nanoparticles. FIG. 2A shows the temperature and pressures used in a lyophilization process that includes an annealing step, in which the composition to be lyophilized is held at −10° C. for 5 hours during freezing. FIG. 2B shows the temperatures and pressures used in a lyophilization process that does not include an annealing step, in which the composition to be lyophilized is directly cooled to −50° C. FIG. 2C shows the delta diameter (left y-axis, data in columns) and percentage encapsulation efficiency (right y-axis, data in lines) of lipid nanoparticles in compositions lyophilized by the processes shown in FIGS. 2A-2B, across a range of sucrose concentrations.

FIG. 3 shows the effect of moisture content on stability of lyophilized compositions comprising nucleic acids. The 1^{st} order rate constant of mRNA degradation (month⁻¹) for mRNA in lyophilized compositions is shown for a range of moisture contents (% w/w).

FIGS. 4A-4B show the effects of secondary drying temperatures on lyophilized compositions comprising nucleic acids. FIG. 4A shows an overview of a lyophilization process comprising a freezing phase with an annealing step, a primary drying phase, and a secondary drying phase. FIG. 4B shows data relating to the moisture content of lyophilized compositions subjected to secondary drying phases with temperatures of 25° C. (squares) or 40° C. (circles) for up to 24 hours. FIGS. 4C-4F show characteristics of lyophilized compositions prepared by secondary drying phases with temperatures of 25° C. (left bar of each group) or 40° C. (right bar of each group). FIG. 4C shows lipid nanoparticle size, as measured by unfiltered dynamic light scattering. FIG. 4D shows percent encapsulation efficiency, as measured by RiboGreen. FIG. 4E shows reduction in mRNA purity. FIG. 4F shows relative in vitro potency. Compositions A and B contained distinct mRNAs.

FIGS. 5A-5D show the effect of moisture content on the stability of mRNA in lyophilized compositions. FIG. 5A shows the 1^{st} order rate constant of mRNA degradation (month⁻¹) for mRNA in lyophilized compositions with various moisture contents (% w/w). FIG. 5B shows the stability over time of mRNA in lyophilized compositions with various moisture contents (% w/w). FIG. 5C shows the stability of mRNA encoding a first protein (Antigen 1) in lyophilized compositions from multiple lots, containing moisture contents between 0.20-0.65 (% w/w), during 6 or 9 months of storage at 5° C. FIG. 5D shows the stability of mRNA encoding a second protein (Antigen 2) in lyophilized compositions from the same lots as FIG. 5C.

FIG. 6 shows the efficiency of expression of a protein (Antigen 3) encoded by mRNA in lyophilized and reconstituted compositions (1^{st}, left group of bars), compositions stored at 4° C. (2^{nd} group), compositions frozen at −20° C. and thawed (3^{rd} group), and compositions frozen at −80° C. and thawed (4^{th} group) In each group, compositions were diluted to deliver varying amounts of mRNA, shown on the x-axis. The height of each bar shows the geometric mean fluorescence intensity of cells stained with Antigen 3-specific antibodies and analyzed by flow cytometry. NT=no treatment (no RNA delivered to cells).

FIG. 7 shows Antigen 3-specific IgG titers in sera of mice that were administered one or two doses of lyophilized or frozen/thawed compositions containing antigen-encoding mRNA. Doses were administered on day 0 (prime) and day 22 (boost), with sera collected on day 21 (3 weeks post-prime dose, before boost) and 36 (2 weeks post-boost dose), and antigen-specific IgG titers were measured by ELISA.

### DETAILED DESCRIPTION

### Methods for Lyophilization

Some aspects of the disclosure relate to methods of lyophilizing nucleic acids, such as mRNA, which can produce enhanced compositions for use as pharmaceuticals. In some embodiments, the nucleic acid is formulated with a lipid, e.g., an ionizable lipid (e.g., in a lipid nanoparticle (LNP)). One issue that arises in lyophilized compositions is the presence of water, which can facilitate the degradation of nucleic acids via hydrolysis. Some embodiments comprise minimizing the moisture content of a lyophilized composition, thus producing lyophilized compositions in which nucleic acids are stable during long-term storage.

It has been discovered that the introduction of an annealing step, in which lipid nanoparticles were held at a temperature near the freezing point of water prior to deep freezing, allowed for the use of a higher temperature during the secondary drying phase without compromising nucleic acid integrity. Furthermore, the inclusion of high levels of sucrose as a lyoprotectant unexpectedly resulted in a reduction in LNP size, increasing the efficiency of the LNP-mRNA production process. The mRNA in the resulting lyophilized compositions thus remains stable during extended storage.

In certain embodiments, the pharmaceutical compositions may be characterized as being stable relative to an equivalent composition prepared by a lyophilization step that does not involve an annealing step, a high level of lyoprotectant, and/or high drying temperatures. The stability of the lyophilized product may be determined, for instance, with reference to the particle size of the lipid nanoparticles comprising such composition. In certain embodiments, lyophilization of the lipid nanoparticles produces a smaller particle size of the lipid nanoparticles following lyophilization and/or reconstitution.

Some embodiments comprise lyophilizing a composition in a system that comprises a refrigeration system, a vacuum system, and a condenser system. In some embodiments, the lyophilization is in a freeze dryer.

### Thermal Treatment

In some embodiments, the lyophilization comprises one or more thermal treatment steps. In some embodiments, the thermal treatment comprises one or more freezing steps (e.g., a first freezing step, a second freezing step, etc.). In some embodiments, the thermal treatment step(s) are performed at atmospheric pressure.

In some embodiments, the thermal treatment comprises an annealing step, in which the temperature of the composition is cycled between two or more temperatures (e.g., a first temperature and a second temperature).

In some embodiments the annealing step comprises cooling the composition to a temperature that is higher than the freezing temperature of the composition prior to being solidified by freezing at a lower temperature. That is, in some embodiments the annealing comprises exposing the composition to a first temperature above the freezing temperature and then exposing the composition to a second temperature below the freezing temperature of the composition.

In some embodiments, the annealing step comprises exposing the composition to a first temperature below the freezing temperature of the composition, a second temperature (e.g., an annealing temperature) above the freezing temperature of the composition, and a third temperature below the freezing temperature of the composition. That is, in some embodiments, the first and third temperatures (e.g., freezing temperatures) are below the freezing temperature of the composition and the second temperature (e.g., annealing temperature) is above the freezing temperature of the composition.

In some embodiments, the annealing temperature that is above the freezing temperature of the composition is a temperature of from about −30° C. to about 0° C., such as about −30° C., about −25° C., about −20° C., about −15° C., about −10° C., about −5° C., or about 0° C.

In some embodiments, the temperature that is below the freezing temperature of the composition is a temperature of from about −100° C. to about −30° C., such as about −100° C., about −90° C., about −80° C., about −70° C., about −60° C., about −50° C., about −40° C., or about −30° C.

In some embodiments, the annealing step is conducted for at least about 30 minutes, such as about 1 hour, about 2 hours, such as about 3 hours, about 4 hours, about 5 hours, about 10 hours, about 15 hours, or about 20 hours or more. In some embodiments, the annealing comprises exposing the composition to two or more temperatures (e.g., a first freezing temperature, a second annealing temperature, a third freezing temperature, etc.), wherein the composition is exposed to each of the various temperatures for a period of about 30 minutes, about 1 hour, about 2 hours, about 3 hours about 4 hours, about 5 hours, about 6 hours, about 8 hours, or about 10 hours or more. In some embodiments, the composition is exposed to each of the temperatures for the same time periods (i.e., the first, second, etc. time periods are the same). In some embodiments, the composition is exposed to each of the temperatures for different time periods (e.g., one or more of the first, second, third, etc. temperatures are for a period of time that is different from the others).

Compared to cooling the composition directly to the final freezing temperature, an annealing step, without being bound by theory, is believed to allow water molecules to form a more ordered arrangement prior to freezing, resulting in the formation of larger ice crystals in the frozen composition. These larger ice crystals may be less likely to damage the nucleic acids of the composition during sublimation or secondary drying. Thus, an annealing step enables the use of harsher conditions during primary and/or secondary drying that may otherwise compromise nucleic acid integrity. Additionally, an annealing step allows for crystallization of bulking agents and/or lyoprotectants (e.g., sucrose), which provide a framework to support the integrity of lyophilized compositions and prevent collapse of a lyophilized composition during primary or secondary drying.

### Sublimation

In some embodiments, the lyophilization comprises a sublimation step (e.g., conducted at temperatures below the compositions critical collapse temperature and performed under a vacuum). In some embodiments, the sublimation occurs in a first drying step. In some embodiments, the sublimation comprises using one or more of conduction, convection, and radiation to provide heat energy sufficient to drive a phase change from solid to gas. Without being bound by theory, it is believed that the sublimation step removes free ice crystals and/or organic solvents in the composition.

In some embodiments, the sublimation comprises exposing a thermally-treated composition to a temperature that is below the melting point of water, but higher than the temperature used to freeze the composition, under vacuum. In some embodiments, the primary drying phase is conducted at a temperature of about −40° C. to about 0° C., such as about −40° C., about −35° C., about −32° C., about −30° C., about −25° C., about −20° C., about −15° C., about −10° C., about −5° C., or about 0° C. In some embodiments, the primary drying phase is conducted at a temperature of from about −35° C. to about −25° C. In some embodiments, the primary drying phase is conducted at a temperature of from about −32 to about −25° C. At low pressure and temperature, ice can sublimate from the composition, such that majority of water is removed. In some embodiments, the primary drying phase is carried for at least 10, at least 20, at least 30, at least 40, at least 50, at least 60, at least 70, or at least 80 hours.

In some embodiments, the sublimation is conducted at a pressure that is 20% to 30% of the vapor pressure of ice at the sublimation temperature. In some embodiments, the sublimation is performed at a vacuum pressure of between about 50 mTorr and about 300 mTorr, such as about 75 mTorr, about 100 mTorr, about 125 mTorr, about 150 mTorr, about 175 mTorr, or about 200 mTorr.

Some embodiments comprise determining that primary drying is complete when a Pirani gauge, which measures the relative vacuum of an environment, outputs a reading at the sublimation temperature that is similar to the reading produced by a capacitance manometer, which measures the absolute pressure of an environment, at the sublimation temperature. A Pirani gauge measures the relative vacuum of an environment using a sensor wire, which is heated by electric current, and measuring the current required to maintain a constant temperature. When more gas is present, a higher a current is required to maintain the temperature. A capacitance manometer measures absolute pressure using a metal diaphragm under tension, with one side of the diaphragm exposed to the gas whose pressure is to be measured, and the other side exposed to a sealed vacuum, which serves as a reference. Pirani gauges are particularly sensitive to the presence of water vapor, and thus produce higher readings than a capacitance manometer when water vapor is still present in the environment, indicating primary drying is not complete. When a Pirani gauge yields a similar measurement to that of a capacitance manometer, which is not sensitive to the presence of water vapor, this convergence indicates a lack of water vapor. This absence of water vapor at the current temperature and pressure indicates that no more ice is being sublimated and primary drying is complete.

### Desorption

In some embodiments, the lyophilization comprises a desorption step. In some embodiments, the desorption occurs in a secondary drying step. In the secondary drying phase, a composition can be warmed to a higher temperature (e.g., as compared to a primary drying step) to facilitate removal of remaining water molecules that are bound to the composition.

In some embodiments, the desorption is conducted at a desorption temperature of at least about 20° C., such as about 30° C., about 35° C., about 40° C., about 45° C., about 50° C., or more. Preferably, the secondary drying phase is conducted at a desorption temperature of about 40° C.

In some embodiments, the secondary drying phase is conducted for at least 30 minutes, at least 1 hour, at least 2 hours, at least 5 hours, at least 10 hours, at least 15 hours, or at least 20 hours.

In some embodiments, the desorption is conducted at a pressure that is 20% to 30% of the vapor pressure of water at the desorption temperature. In some embodiments, the desorption is performed at a vacuum pressure of between about 50 mTorr and about 300 mTorr, such as about 75 mTorr, about 100 mTorr, about 125 mTorr, about 150 mTorr, about 175 mTorr, or about 200 mTorr.

In some embodiments, the secondary drying phase is conducted until a Pirani gauge measuring a relative vacuum at the desorption temperature produces a reading that is about the same as the reading produced by a capacitance manometer measuring absolute pressure at the desorption temperature. Warming the composition to a higher temperature than the temperature used in primary drying promotes the evaporation of residual water from the composition, causing the reading of a Pirani gauge to again become elevated relative to the reading of a capacitance manometer. Convergence of the Pirani gauge reading to that of a capacitance manometer indicates an absence of water vapor, as no more water is being evaporated at the current desorption temperature.

In some embodiments, the secondary drying phase is conducted until the moisture content of the composition is less than about 6% w/w, less than about 5.5% w/w, less than about 5% w/w, less than about 4.5% w/w, less than about 4% w/w, less than about 3.5% w/w, less than about 3% w/w, less than about 2.5% w/w, less than about 2% w/w, less than about 1% w/w, or less than about 0.5% w/w.

### Lyoprotectants

Some aspects of the disclosure relate to compositions (e.g., lipid nanoparticle compositions) and methods of preparing lyophilized compositions, wherein the composition comprises a lyoprotectant.

In some embodiments, the compositions comprise a lipid nanoparticle comprising a nucleic acid, such as mRNA. In some embodiments, a lyoprotectant is added to the nucleic acid (e.g., mRNA) before the nucleic acid is combined with the lipid or lipid nanoparticles and prior to lyophilization, such that the final composition includes the lyoprotectant. In some embodiments, the lyoprotectant is added to the lipid or lipid nanoparticle before the lipid nanoparticle is combined with the nucleic acid. In some embodiments, the lyoprotectant, lipid, and nucleic acid are combined prior to formation of the lipid nanoparticle. In some embodiments, the lyoprotectant is added to a composition comprising a lipid nanoparticle and a nucleic acid.

As used herein, a lyoprotectant refers to a composition that mitigates the effects of lyophilization on product integrity. The processes of thermal treatment, sublimation, and desorption expose a composition to harsh conditions, which may reduce the integrity of one or more components of the lyophilized composition. Inclusion of a lyoprotectant in a composition to be lyophilized may prevent or reduce some of this reduction in integrity through multiple mechanisms of action. For example, without being bound by theory a lyoprotectant may alter the structure of ice crystals in a frozen composition and/or act as a buffer between a composition to be preserved and ice crystals formed during freezing, such that sublimating ice crystals are less likely to damage the composition to be preserved Additionally, a lyoprotectant may slow the rate of temperature increase in a composition during desorption, reducing the likelihood of undesired chemical reactions that could compromise product integrity.

In some embodiments, the lyoprotectant is a sugar. As used herein, a sugar is a carbohydrate molecule comprising one or more monosaccharide monomers. Non-limiting examples of sugars include sucrose, trehalose, maltose, lactose, glucose, fructose, and galactose. In some embodiments, the sugar is sucrose. Sucrose refers to a compound of the formula C₁₂H₂₂O₁₁ that is a dimer of a glucose monomer and a fructose monomer. Trehalose refers to a compound of the formula C₁₂H₂₂O₁₁ that is a dimer of two glucose monomers joined by a 1,1-glycosidic linkage. Maltose refers to a compound of the formula C₁₂H₂₂O₁₁ that is a dimer of two glucose monomers joined by a 1,4-glycosidic linkage Lactose refers to a compound of the formula C₁₂H₂₂O₁₁ that is a dimer of a glucose monomer and a galactose monomer.

In some embodiments, the lyoprotectant is a polyol. In some embodiments, the lyoprotectant is mannitol. In some embodiments, the lyoprotectant is maltitol. In some embodiments, the lyoprotectant is glycine. In some embodiments, the lyoprotectant is cyclodextrin. In some embodiments, the lyoprotectant is maltodextrin.

In some embodiments, the ratio of sugar mass (e.g., sucrose) to lipid mass (e.g., total mass of ionizable lipids, non-cationic lipids, sterols, and PEG-modified lipids) in the composition is about 5:1 to about 40:1 (5 g sugar:1 g lipids to 40 g sugar:1 g lipids). In some embodiments, the ratio of sugar mass to lipid mass is about 8:1 to about 20:1. In some embodiments, the ratio of sugar mass to lipid mass is about 5:1, about 6:1, about 7:1, about 8:1, about 9:1, about 10.1, about 11:1, or about 12.1. In some embodiments, the ratio of sugar mass to lipid mass is at least 8:1. In some embodiments, the ratio of sugar mass to lipid mass is at least 10:1.

In some embodiments, the ratio of lyoprotectant mass to lipid mass in the composition is about 5:1 to about 40:1 (5 g lyoprotectant:1 g lipids to 40 g lyoprotectant:1 g lipids). In some embodiments, the ratio of lyoprotectant mass to lipid mass is about 8:1 to about 20:1. In some embodiments, the ratio of lyoprotectant mass to lipid mass is about 5:1, about 6:1, about 7:1, about 8:1, about 9:1, about 10:1, about 11:1, or about 12:1. In some embodiments, the ratio of lyoprotectant mass to lipid mass is at least 8:1. In some embodiments, the ratio of lyoprotectant mass to lipid mass is at least 10:1.

In some embodiments, the ratio of the sugar mass to a lipid nanoparticle mass is from about 5:1 to about 40:1. In some embodiments, the ratio of sugar mass to lipid nanoparticle mass is about 8:1 to about 20:1. In some embodiments, the ratio of sugar mass to lipid nanoparticle mass is about 5:1, about 6:1, about 7:1, about 8:1, about 9:1, about 10:1, about 11:1, or about 12:1. In some embodiments, the ratio of sugar mass to lipid nanoparticle mass is at least 8:1. In some embodiments, the ratio of sugar mass to lipid nanoparticle mass is at least 10:1.

In some embodiments, the composition contains at least about 5% w/w sucrose, such as about 6% w/w, about 7% w/w, about 8% w/w, about 9% w/w, about 10% w/w, about 11% w/w, about 12% w/w, about 15% w/w, or more sucrose.

In some embodiments, the sucrose is added to a nucleic acid (e.g., mRNA) before the nucleic acid is combined with a lipid or lipid nanoparticles and prior to lyophilization, such that the final composition includes the sucrose. In some embodiments, the sucrose is added to the lipid or lipid nanoparticle before the lipid nanoparticle is combined with the nucleic acid. In some embodiments, the sucrose, lipid, and nucleic acid are combined prior to formation of the lipid nanoparticle. In some embodiments, the sucrose is added to a composition comprising a lipid nanoparticle and a nucleic acid.

### Buffers and Composition Components

Some aspects of the disclosure relate to compositions (e.g., lipid nanoparticle compositions) and methods of preparing lyophilized compositions, wherein the composition comprises a buffer and/or other components. In some embodiments, the buffer and/or other components are present in pre-lyophilized compositions. In some embodiments, the buffer and/or other components are present in reconstituted lyophilized compositions (e.g., they can be used to reconstitute a lyophilized composition).

In some embodiments, the compositions comprise a lipid nanoparticle comprising a nucleic acid, such as mRNA, and a buffer. In some embodiments, a buffer is added to the nucleic acid (e.g., mRNA) before the nucleic acid is combined with the lipid or lipid nanoparticles and prior to lyophilization, such that the final composition includes the buffer. In some embodiments, the buffer is added to the lipid or lipid nanoparticle before the lipid nanoparticle is combined with the nucleic acid. In some embodiments, the buffer, lipid, and nucleic acid are combined prior to formation of the lipid nanoparticle. In some embodiments, the buffer is added to a composition comprising a lipid nanoparticle and a nucleic acid.

As used herein, a buffer refers to a composition that mitigates the effects of acid or bases on the pH of a composition containing the buffer. The processes of thermal treatment, sublimation, and desorption alter the temperature and amount of water in a composition, both of which may change the pH of the composition and facilitate undesired chemical reactions that compromise the integrity of the composition. Inclusion of a buffer in a composition to be lyophilized may prevent or reduce some of this reduction in integrity through multiple mechanisms of action. For example, without being bound by theory a buffer may absorb free hydrogen or hydroxide ions that are released from other components of the composition before, during, or after lyophilization. Absorption of free hydrogen and/or hydroxide ions by a buffer prevents the ions from reacting with other components of the composition, such as nucleic acids or lipids, which may otherwise facilitate nucleic acid cleavage, modification of nucleic acid structure, or disruption of lipid nanoparticle integrity. Additionally, a buffer may slow the rate of pH change in a composition after lyophilization, reducing the likelihood of undesired pH change that could compromise product integrity.

In some embodiments, the LNP, nucleic acid, or LNP-encapsulated nucleic acid is exposed to a lyophilization buffer via TFF diafiltration. In some embodiments, the LNP, nucleic acid, or LNP-encapsulated nucleic acid is exposed to a lyophilization buffer via dialysis. the LNP, nucleic acid, or LNP-encapsulated nucleic acid is exposed to a lyophilization buffer via a combination of TFF diafiltration and dialysis.

In some embodiments, the buffer is selected from the group consisting of a Tris buffer, citrate buffer, phosphate buffer, triethylammonium bicarbonate (TEAB), and histidine buffer. In some embodiments, the buffer is a Tris buffer. In some embodiments, the buffer is a citrate buffer. In some embodiments, the buffer is a phosphate buffer. In some embodiments, the buffer is a TEAB buffer. In some embodiments, the buffer is a histidine buffer.

In some embodiments, the concentration of the buffer in the composition prior to lyophilization is about 1 mM to about 100 mM. In some embodiments, the concentration of the buffer in the composition prior to lyophilization is about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM. In some embodiments, the concentration of the buffer in the composition prior to lyophilization is about 10 mM. In some embodiments, the concentration of the buffer in the composition prior to lyophilization is about 20 mM. In some embodiments, the concentration of the buffer in the composition prior to lyophilization is about 25 mM. In some embodiments, the concentration of the buffer in the composition prior to lyophilization is about 30 mM. In some embodiments, the buffer concentration is about 100 mM or less, such as about 75 mM or less, about 50 mM or less, about 25 mM or less, or about 10 mM or less. In some embodiments, the buffer concentration is from about 5 mM to about 100 mM, such as about 10 mM to about 50 mM, about 5 mM to about 20 mM. or about 20 mM to about 30 mM.

In some embodiments, the concentration of the buffer in the composition after lyophilization and reconstitution is about 1 mM to about 100 mM. In some embodiments, the concentration of the buffer in the composition after lyophilization and reconstitution is about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM. In some embodiments, the concentration of the buffer in the composition after lyophilization and reconstitution is about 10 mM. In some embodiments, the concentration of the buffer in the composition after lyophilization and reconstitution is about 20 mM. In some embodiments, the concentration of the buffer in the composition prior to lyophilization is about 25 mM. In some embodiments, the concentration of the buffer in the composition after lyophilization and reconstitution is about 30 mM. In some embodiments, the buffer concentration is about 100 mM or less, such as about 75 mM or less, about 50 mM or less, about 25 mM or less, or about 10 mM or less. In some embodiments, the buffer concentration is from about 5 mM to about 100 mM, such as about 10 mM to about 50 mM, about 5 mM to about 20 mM. or about 20 mM to about 30 mM. In some embodiments, the pH of the composition prior to lyophilization is about 6 to about 9. In some embodiments, the pH is about 6 to about 7. In some embodiments, the pH is about 6.0, about 6.1, about 6.2, about 6.3, about 6.4, about 6.5, about 6.6, about 6.7, about 6.8, about 6.9, or about 6.0. In some embodiments, the pH is about 7 to about 8. In some embodiments, the pH is about 7.0, about 7.1, about 7.2, about 7.3, about 7.4, about 7.5, about 7.6, about 7.7, about 7.8, about 7.9 or about 8.0. In some embodiments, the pH is about 8 to about 9. In some embodiments, the pH is about 8.0, about 8.1, about 8.2, about 8.3, about 8.4, about 8.5, about 8.6, about 8.7, about 8.8, about 8.9, or about 9.0. In some embodiments, the pH is about 7.0 to about 7.2, about 7.2 to about 7.4, about 7.4 to about 7.6, about 7.6 to about 7.8, or about 7.8 to about 8.0.

In some embodiments, the pH of the composition after lyophilization and reconstitution is about 6 to about 9. In some embodiments, the pH is about 6 to about 7. In some embodiments, the pH is about 6.0, about 6.1, about 6.2, about 6.3, about 6.4, about 6.5, about 6.6, about 6.7, about 6.8, about 6.9, or about 6.0. In some embodiments, the pH is about 7 to about 8. In some embodiments, the pH is about 7.0, about 7.1, about 7.2, about 7.3, about 7.4, about 7.5, about 7.6, about 7.7, about 7.8, about 7.9 or about 8.0. In some embodiments, the pH is about 8 to about 9. In some embodiments, the pH is about 8.0, about 8.1, about 8.2, about 8.3, about 8.4, about 8.5, about 8.6, about 8.7, about 8.8, about 8.9, or about 9.0. In some embodiments, the pH is about 7.0 to about 7.2, about 7.2 to about 7.4, about 7.4 to about 7.6, about 7.6 to about 7.8, or about 7.8 to about 8.0.

In some embodiments, the pH is below the pKa of an amino lipid in an ionizable lipid in a LNP, such as about 0.2, about 0.3, about 0.4, about 0.5, about 0.6, about 0.7, about 0.8, about 0.9, about 1, about 1.1, about 1.2, about 1.3, about 1.4, about 1.5, about 2, or more pH units below the pKa of an amino lipid in the ionizable lipid.

In some embodiments, the composition (e.g., prior to lyophilization) comprises a salt, such as sodium chloride. In some embodiments, the salt concentration in the composition is from about 0.1 mM to about 300 mM. Preferably, the salt concentration in a pre-lyophilized composition is about 50 mM or less, such as from about 0 mM to about 50 mM, or about 0.1 mM to about 50 mM. In a reconstitution medium, the salt concentration is preferably from about 25 mM to about 200 mM, such as about 25 mM, about 50 mM, about 75 mM, about 100 mM, about 125 mM, about 150 mM, about 175 mM, or about 200 mM.

In some embodiments, the composition (e.g., prior to lyophilization) comprises a surfactant. Exemplary surfactants include Tween20, Tween80, BrijS200, and PEG-DMG. In some embodiments, the composition comprises from about 0.0001 wt % to about 0.5 wt % surfactant, such as about 0.001 wt % to about 0.4 wt %, about 0.002 wt % to about 0.3 wt %, about 0.003 wt % to about 0.2 wt %, or about 0.005 wt % to about 0.1 wt % surfactant. In some embodiments, the composition comprises about 0.005 wt %, about 0.05 wt %, about 0.01 wt %, about 0.1 wt %, or about 0.5 wt % surfactant. In some embodiments, the composition (e.g., prior to lyophilization) comprises a metal chelator. Exemplary metal chelators include EDTA (ethylenediaminetetraacetic acid) and DTPA (diethylenetriaminepentaacetic acid). In some embodiments, the composition comprises from 0.1 mM to about 5 mM or more of the metal chelator, such as about 0.5 mM, about 0.75 mM, about 1 mM, about 1.5 mM, about 2 mM, about 2.5 mM, about 3 mM, about 3.5 mM, about 4 mM, about 4.5 mM, or about 5 mM of the metal chelator.

### Lipid Compositions

In some embodiments, the nucleic acids are formulated as a lipid composition, such as a composition comprising a lipid nanoparticle, a liposome, and/or a lipoplex. In some embodiments, nucleic acids are formulated as lipid nanoparticle (LNP) compositions. Lipid nanoparticles typically comprise amino lipid, non-cationic lipid, structural lipid, and PEG lipid components along with the nucleic acid cargo of interest. The lipid nanoparticles can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid, 5-25% non-cationic lipid, 25-55% structural lipid, and 0.5-15% PEG-modified lipid.

In some embodiments, the lipid nanoparticle comprises a molar ratio of 20-60% ionizable amino lipid, 5-30% non-cationic lipid, 10-55% structural lipid, and 0.5-15% PEG-modified lipid.

In some embodiments, the lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, the lipid nanoparticle comprises 20-60 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, the lipid nanoparticle comprises 45-55 mole percent (mol %) ionizable amino lipid. For example, lipid nanoparticle may comprise 45, 46, 47, 48, 49, 50, 51, 52, 53, 54, or 55 mol % ionizable amino lipid.

### Ionizable Amino Lipids

In some embodiments, the ionizable amino lipid is a compound of Formula (AI):

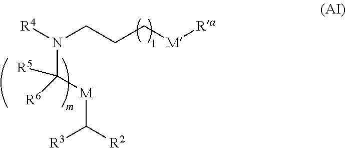

or its N-oxide, or a salt or isomer thereof,
wherein R′^{a} is R′^{branched}; wherein
R′^{branched} is:

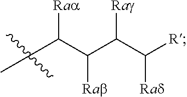

wherein

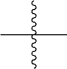

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

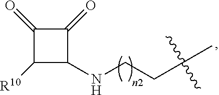

wherein

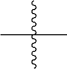

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

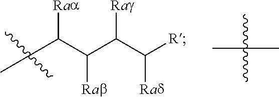

denotes a point of attachment; R^{aα}, R^{aβ}, R^{aγ}, and R^{aδ} are each H; R² and R³ are each C₁₋₁₄ alkyl; R⁴ is —(CH)ₙOH, n is 2; each R⁵ is H; each R⁶ is H; M and M′ are each —C(O)O—; R′ is a C₁₋₁₂ alkyl; l is 5; and m is 7.

In some embodiments of the compounds of Formula (AI), R′^{a} is R′^{branched}; R′^{branched} is

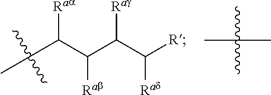

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of the compounds of Formula (AI), R′^{a} is R′^{branched}; R′^{branched} is

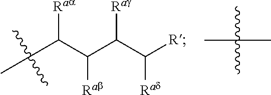

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

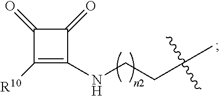

R¹⁰ NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}; R′^{branched} is

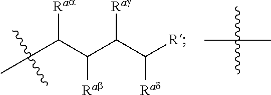

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the compound of Formula (I) is selected from:

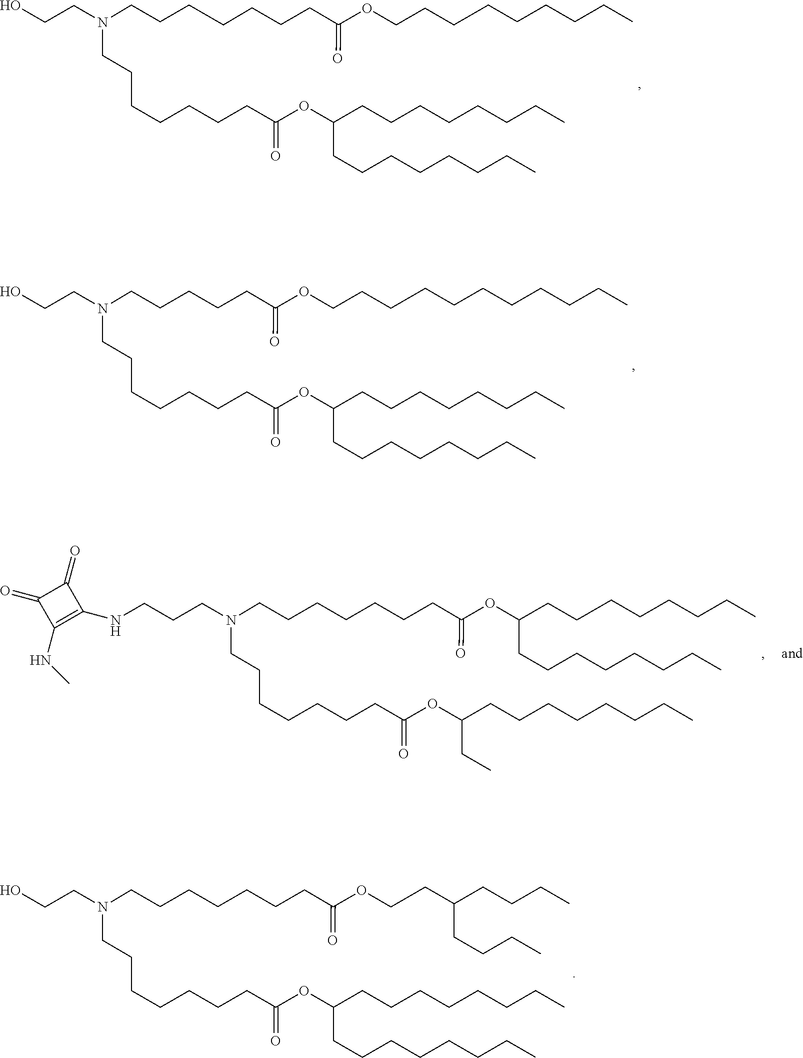 [SMILES: CCCCCCCCCCOC(=O)CCCCCCCCN(CCO)CCCCCCCC(=O)OC(CCCCCCCC)CCCCCCCC ; CCCCCCCCCCCCOC(=O)CCCCCN(CCO)CCCCCCCC(=O)OC(CCCCCCCC)CCCCCCCC ; CCCCCCCCCC(CCCCCCCC)OC(=O)CCCCCCCN(CCCCCCCC(=O)OC(CC)CCCCCCCC)CCCNc1c(NC)c(=O)c1=O ; CCCCCCCCC(CCCCCCCC)OC(=O)CCCCCCCN(CCO)CCCCCCCC(=O)OCCCC(CCCC)CCCC]

In some embodiments, the ionizable amino lipid is a compound of Formula (Ala):

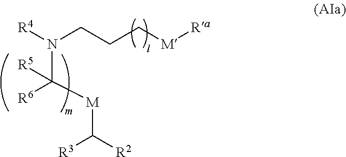

or its N-oxide, or a salt or isomer thereof,
wherein R′^{a} is R′^{branched}; wherein
R′^{branched} is:

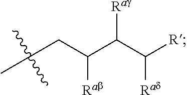

wherein

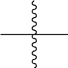

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

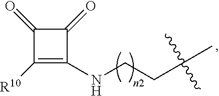

wherein

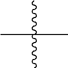

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid is a compound of Formula (AIb):

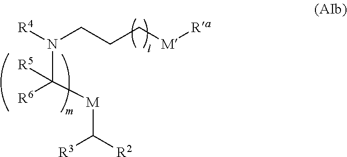

or its N-oxide, or a salt or isomer thereof,
wherein R′^{a} is R′^{branched}; wherein
R′^{branched} is:

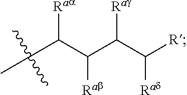

wherein

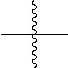

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

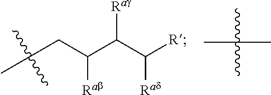

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of Formula (AI) or (AIb), R′^{a} is R′^{branched}; R′^{branched} is

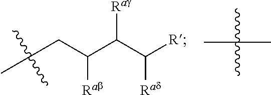

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of Formula (AI) or (AIb), R′^{a} is R′^{branched}; R′^{branched} is

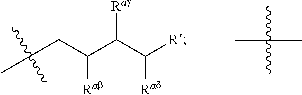

denotes a point of attachment; R^{aβ} and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the ionizable amino lipid is a compound of Formula (AIc):

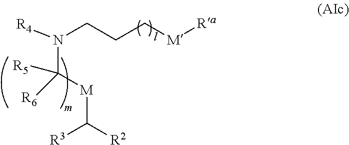

or its N-oxide, or a salt or isomer thereof,
wherein R′^{a} is R′^{branched}; wherein
R′^{branched} is:

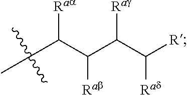

wherein

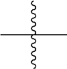

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

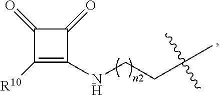

wherein

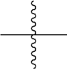

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

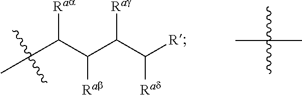

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

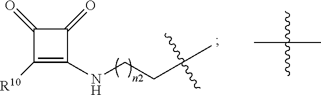

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (AIc) is:

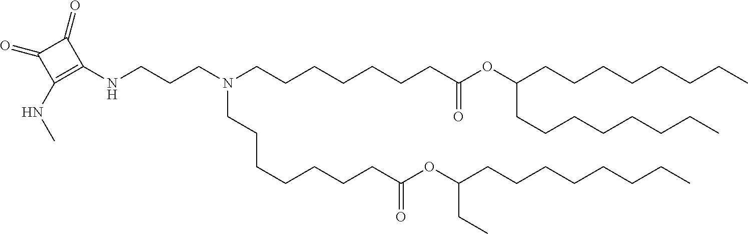 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

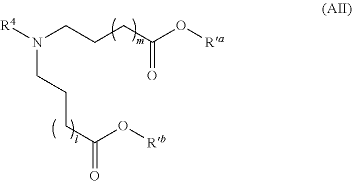

or its N-oxide, or a salt or isomer thereof,
wherein R′^{a} is R′^{branched} or R′^{cyclic}; wherein
R′^{branched} is:

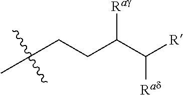

and R′^{cyclic} is:

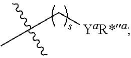

and
R′^{b} is:

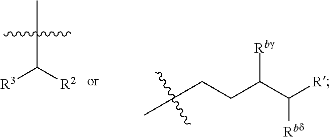

wherein

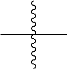

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

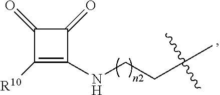

wherein

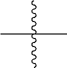

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-a):

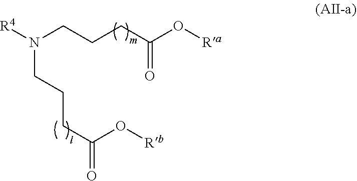

or its N-oxide, or a salt or isomer thereof,
wherein R′^{a} is R′^{branched} or R′^{cyclic}; wherein
R′^{branched} is:

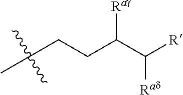

and R′^{b} is:

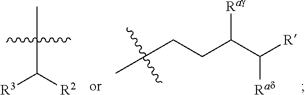

wherein

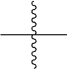

denotes a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

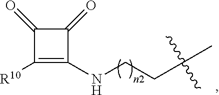

wherein

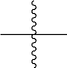

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-b):

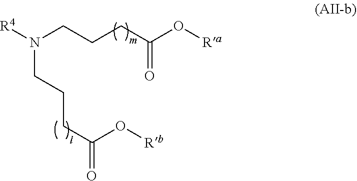

or its N-oxide, or a salt or isomer thereof,
wherein R′^{a} is R′^{branched} or R′^{cyclic}; wherein
R′^{branched} is:

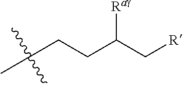

and R′^{b} is:

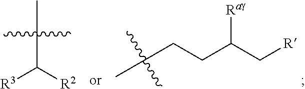

wherein

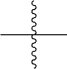

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

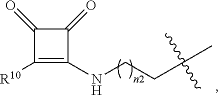

wherein

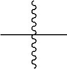

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-c):

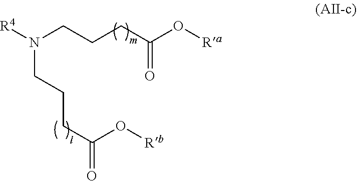

or its N-oxide, or a salt or isomer thereof,
wherein R′^{a} is R′^{branched} or R′^{cyclic}; wherein
R′^{branched} is:

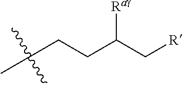

and R′^{b} is:

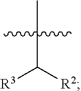

wherein

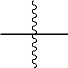

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

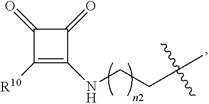

wherein

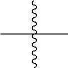

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-d):

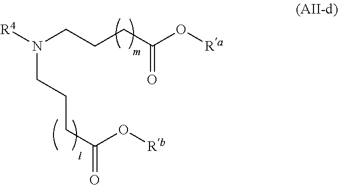

or its N-oxide, or a salt or isomer thereof,
wherein R′^{a} is R′^{branched} or R′^{cyclic}; wherein
R′^{branched} is:

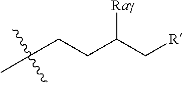

and R′^{b} is:

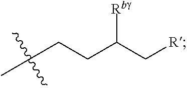

wherein

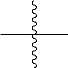

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

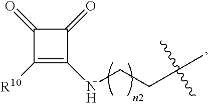

wherein

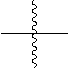

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-e):

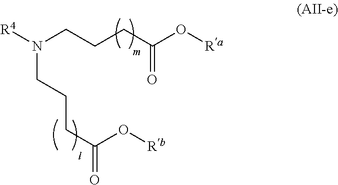

or its N-oxide, or a salt or isomer thereof,
wherein R′^{a} is R′^{branched} or R′^{cyclic}; wherein
R′^{branched} is:

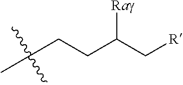

and R′^{b} is:

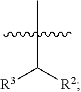

wherein

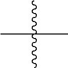

denotes a point of attachment; wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

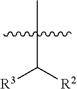

and R² and R³ are each independently a C₁₋₁₄ alkyl. In some embodiments of the compound of Formula (AII), (AII-a), (AII-b), (AII-c), (AII-d), or (AII-e), R′^{b} is:

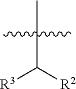

and R² and R³ are each independently a C₆₋₁₀ alkyl. In some embodiments of the compound of Formula (AII), (AII-a), (AII-b), (AII-c), (AII-d), or (AII-e), R′^{b} is:

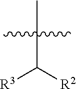

and R² and R³ are each a Ca alkyl.

In some embodiments of the compound of Formula (AII), (AII-a), (AII-b), (AII-c), (AII-d), or (AII-e), R′^{branched} is:

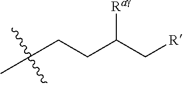

### and R′^{b} is:

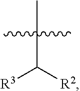

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

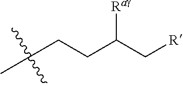

### and R′^{b} is:

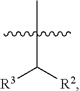

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

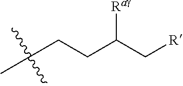

### and R′^{b} is:

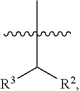

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

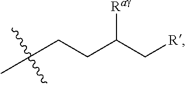

### R′^{b} is:

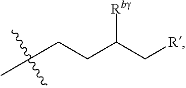

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

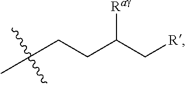

### R′^{b} is:

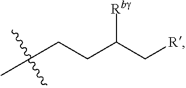

and R^{aγ} and R^{bγ} are each a C₂₋₆ alkyl.

In some embodiments of the compound of Formula (All), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (AII), (AII-a), (AII-b), (AII-c), (AII-d), or (AII-e), R′^{branched} is:

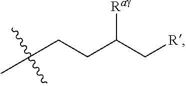

### R′^{b} is:

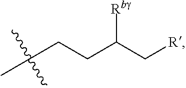

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

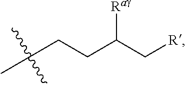

### R′^{b} is:

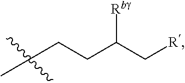

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

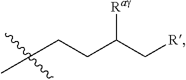

### and R′^{b} is:

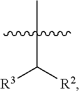

m and l are each independently selected from 4, 5, and 6, R′ is a C₁₋₁₂ alkyl, R^{aγ} is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

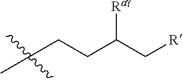

### and R′^{b} is:

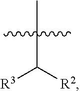

m and l are each 5, R′ is a C₂₋₅ alkyl, R^{aγ} is a C₂₋₆ alkyl, and R² and R³ are each a C₈ alkyl.

In some embodiments of the compound of (AII), (AII-a), (AII-b), (AII-c), (AII-d), or (AII-e), R⁴ is

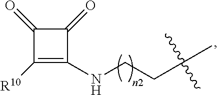

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

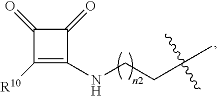

wherein R¹⁰ is NH(CH₃) and n2 is 2.

In some embodiments of the compound of Formula (AII), (AII-a), (AII-b), (AII-c), (AII-d), or (AII-e), R′^{branched} is:

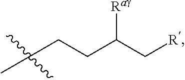

### R′^{b} is:

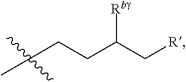

m and l are each independently selected from 4, 5, and 6, each R′ independently is a C₁₋₁₂ alkyl, R^{aγ} and R^{bγ} are each a C₁₋₁₂ alkyl, and R⁴ is

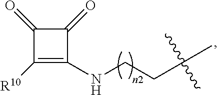

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

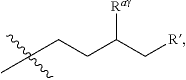

### R′^{b} is:

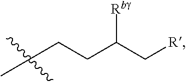

m and l are each 5, each R′ independently is a C₂₋₅ alkyl, R^{aγ} and R^{bγ} are each a C₂₋₆ alkyl, and R⁴ is

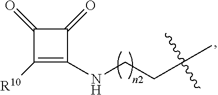

wherein R¹⁰ is NH(CH₃) and n2 is 2.

In some embodiments of the compound of Formula (AII), (AII-a), (AII-b), (AII-c), (AII-d), or (AII-e), R′^{branched} is:

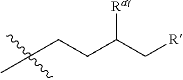

### and R′^{b} is:

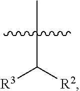

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

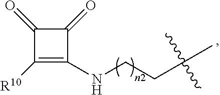

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

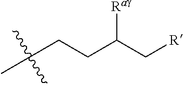

### and R′^{b} is

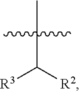

m and l are each 5, R′ is a C₂₋₅ alkyl, R^{aγ} is a C₂₋₆ alkyl, R² and R³ are each a C₈ alkyl, and R⁴ is

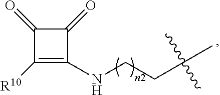

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂) OH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

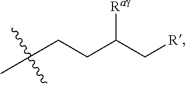

### R′^{b} is

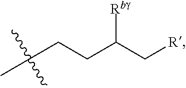

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

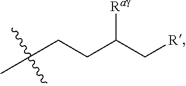

### R′^{b} is:

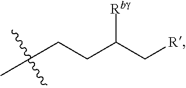

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

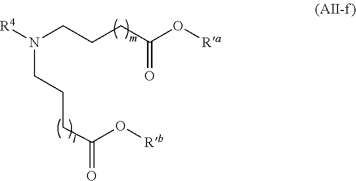

or its N-oxide, or a salt or isomer thereof,
wherein R′^{a} is R′^{branched} or R′^{cyclic}; wherein
R′^{branched} is:

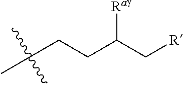

and R′^{b} is:

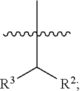

wherein

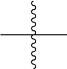

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid is a compound of Formula (AII-g):

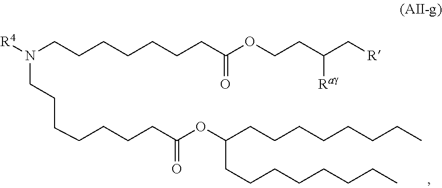

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

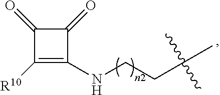

wherein

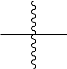

denotes a point of attachment, R¹⁰ is NH(C₁₋₆ alkyl), and n2 is selected from the group consisting of 1, 2, and 3.

In some embodiments, the ionizable amino lipid is a compound of Formula (AII-h):

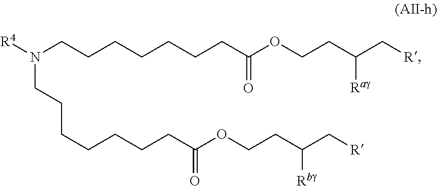

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

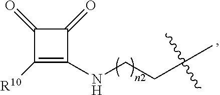

wherein

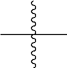

wherein denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

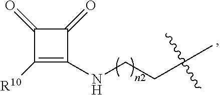

wherein
R¹⁰ is NH(CH₃) and n2 is 2.

In some embodiments of the compound of Formula (AII-g) or (AII-h), R⁴ is —(CH₂)₂OH.

In some embodiments, the ionizable amino lipids may be one or more of compounds of Formula (VI):

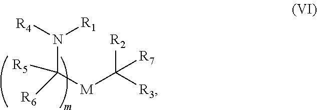

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I, and
m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I, and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′,
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₅ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (VI) includes those of Formula (VI-A):

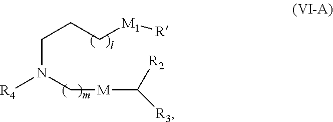

or its N-oxide, or a salt or isomer thereof, wherein l is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₅ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VI-B):

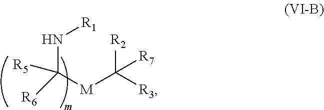

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₅ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

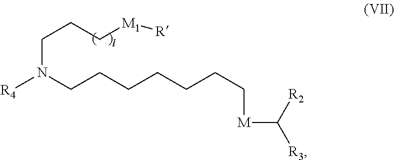

or its N-oxide, or a salt or isomer thereof, wherein l is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (VI) are of Formula (VIIa),

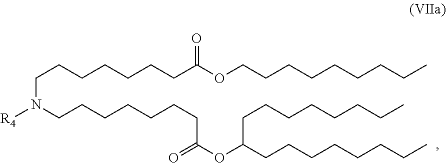

or their N-oxides, or salts or isomers thereof, wherein R₄ is as described herein.

In another embodiment, the compounds of Formula (VI) are of Formula (VIIb),

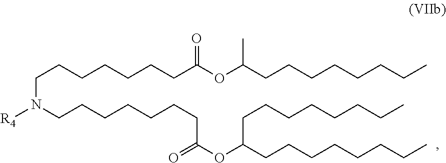

or their N-oxides, or salts or isomers thereof, wherein R₄ is as described herein.

In another embodiment, the compounds of Formula (VI) are of Formula (VIIc) or (VIIe):

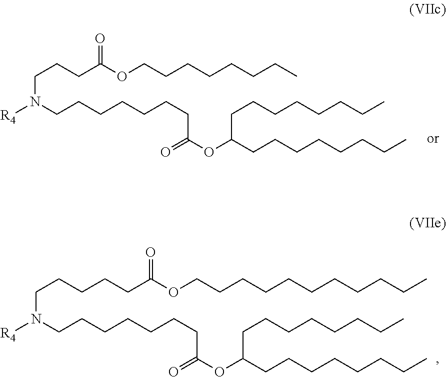

or their N-oxides, or salts or isomers thereof, wherein R₄ is as described herein.

In another embodiment, the compounds of Formula (VI) are of Formula (VIIf):

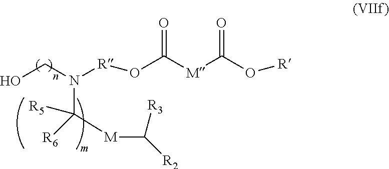

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

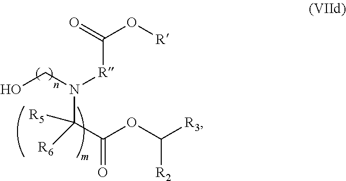

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid comprises a compound having structure:

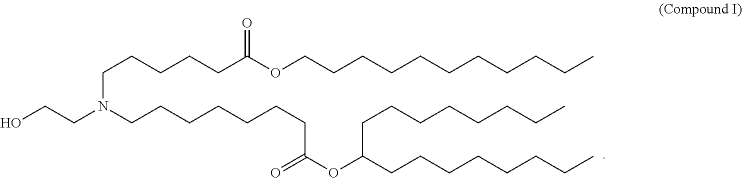 [SMILES: CCCCCCCCCCCOC(=O)CCCCCN(CCO)CCCCCCCC(=O)OC(CCCCCCCC)CCCCCCCC]

In some embodiments, an ionizable amino lipid comprises a compound having structure:

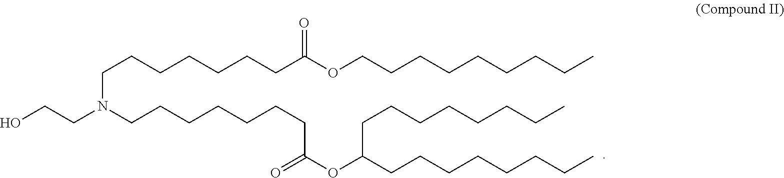 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

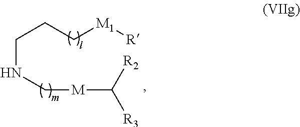

or their N-oxides, or salts or isomers thereof, wherein l is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipids may be one or more of compounds of formula (VIII),

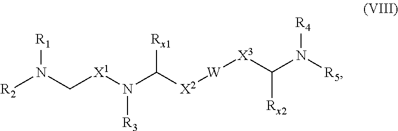

or salts or isomers thereof, wherein
W is

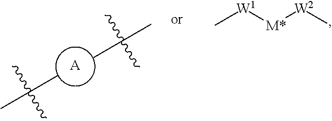

ring A is

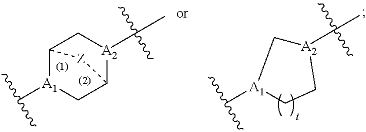

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O)—, —S(O)₂—, —C(O)S—, —SC(O)—, an aryl group, and a heteroaryl group;
M* is C₁-C₆ alkyl,
W¹ and W² are each independently selected from the group consisting of —O— and —N(R₆)—;
each R₆ is independently selected from the group consisting of H and Ct-s alkyl;
X¹, x², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl and —R*MR′; and
n is an integer from 1-6;
wherein when ring A is

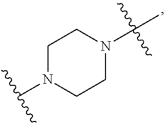

then
i) at least one of X¹, X², and X³ is not —CH₂—; and/or
ii) at least one of R₁, R₂, R₃, R₄, and R₅ is —R″MR′.

In some embodiments, the compound is of any of formulae (VIIIa1)-(VIIIa8):

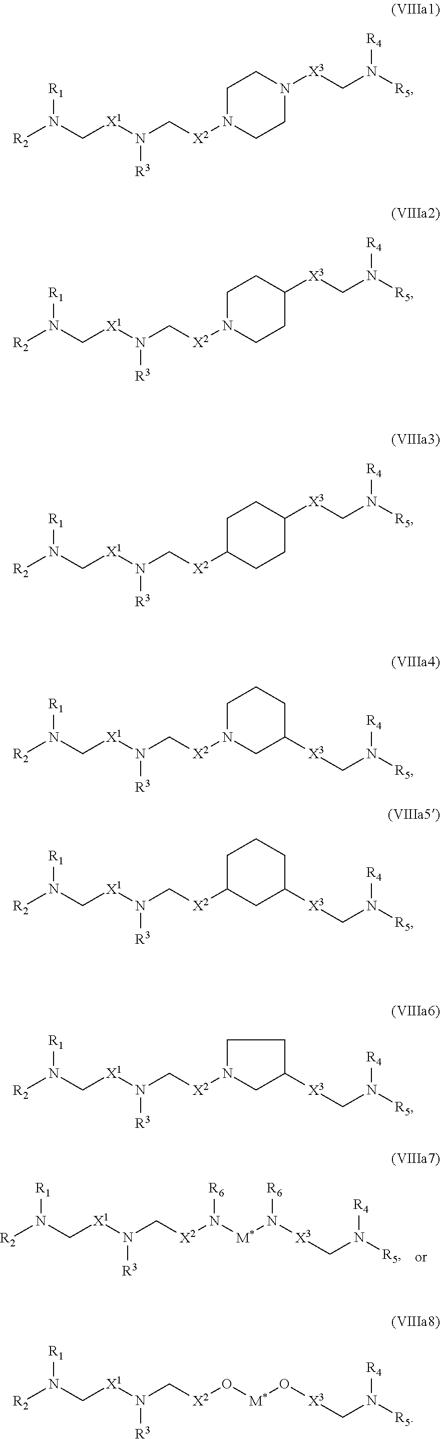

In some embodiments, the ionizable amino lipid is

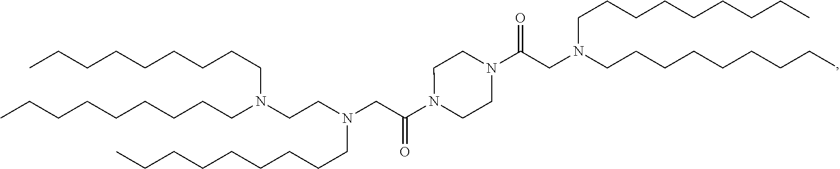

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, the lipid nanoparticle comprises a lipid having the structure:

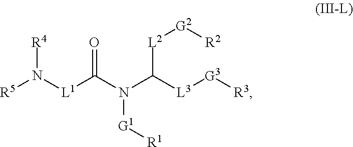

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In some embodiments, the lipid nanoparticle comprises a lipid having the structure:

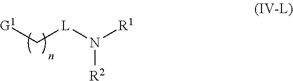

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and
n is an integer from 1 to 12.

In some embodiments, the lipid nanoparticle comprises a lipid having the structure:

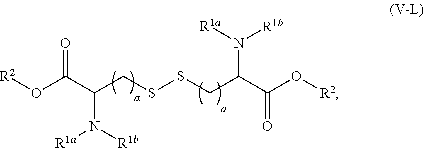

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each I.₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

In some embodiments, the lipid nanoparticle comprises a lipid having the structure:

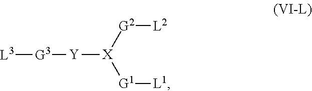

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
L³ is —O(C═O)R³ or —(C═O)OR³,
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

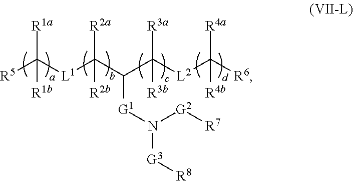

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-ₛ-S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C—C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4A} and R^{4B} are, at each occurrence, independently either: (a) H or C₁-C₁₂ alkyl; or (b) R^{4A} is H or C₁-C₁₂ alkyl, and R^{4B} together with the carbon atom to which it is bound is taken together with an adjacent R^{4B} and the carbon atom to which it is bound to form a carbon-carbon double bond;
R⁵ and R⁶ are each independently H or methyl;
R⁷ is H or C—C₂₀ alkyl;
R⁸ is OH, —N(R⁹)(C═O)R¹⁰, —(C═O)NR⁹R¹⁰, —NR⁹R¹⁰, —(C═O)OR″¹ or —O(C═O)R″, provided that G³ is C₄-C₆ alkylene when R⁸ is —NR⁹R¹⁰,
R⁹ and R¹⁰ are each independently H or C₁-C₁₂ alkyl;
R″ is aralkyl;
a, b, c and d are each independently an integer from 1 to 24; and x is 0, 1 or 2, wherein each alkyl, alkylene and aralkyl is optionally substituted.

In some embodiments, the lipid nanoparticle comprises a lipid having the structure:

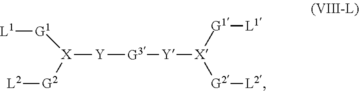

or a pharmaceutically acceptable salt, prodrug or stereoisomer thereof, wherein:
X and X′ are each independently N or CR;
Y and Y′ are each independently absent, —O(C═O)—, —(C═O)O— or NR, provided that:
   a) Y is absent when X is N;
   b) Y′ is absent when X′ is N;
   c) Y is —O(C═O)—, —(C═O)O— or NR when X is CR; and
   d) Y′ is —O(C═O)—, —(C═O)O— or NR when X′ is CR,
L¹ and L^{1′} are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L^{2′} are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹. G^{1′}, G² and G^{2′} are each independently C₂-Ci₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

In some embodiments, the lipid nanoparticle comprises a lipid having the structure:

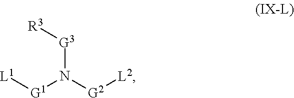

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, the lipid nanoparticle comprises a lipid having the structure:

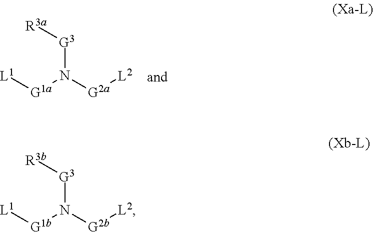

or a pharmaceutically acceptable salt, prodrug or stereoisomer thereof, wherein:
L¹ is —O(C═O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f},
—OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈ alkyl or C₂-C₈ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In some embodiments, the lipid nanoparticle comprises a lipid having the structure:

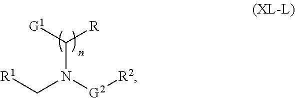

or a pharmaceutically acceptable salt, prodrug or stereoisomer thereof, wherein:
G¹ is —OH, —R³R⁴, —(C═O)R⁵ or —R³(C═O)R₅;
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated Ci-C₆ alkyl; and
n is an integer from 2 to 6.

In some embodiments, the lipid nanoparticle comprises a lipid having the structure:

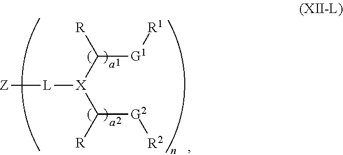

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ˜O(C═O)—, wherein ˜ represents a covalent bond to X; X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

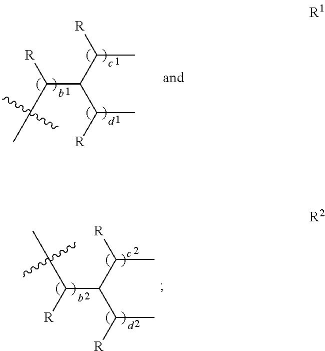

a¹ and a² are, at each occurrence, independently an integer from 3 to 12; b¹ and b² are, at each occurrence, independently 0 or 1;
c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6,
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, the lipid nanoparticle comprises a lipid having the structure:

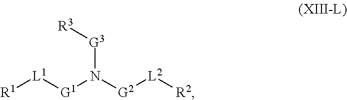

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O) R^{a}—, —OC(═O) R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O) R^{a}—, R^{a}C(═O) R^{a}—, —OC(═O) R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴;
R⁴ is C₁-C₁₂ alkyl;
R⁵ is H or C₁-C₆ alkyl; and
x is 0, 1 or 2.

In some embodiments, the lipid nanoparticle comprises a lipid having the structure:

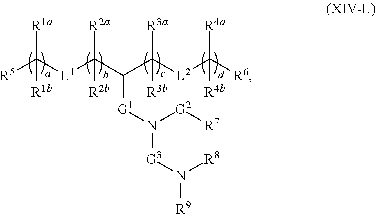

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O) R^{a}—, —R^{a}C(═O)R^{a}—, —OC(═O)R^{a}—, —R^{a}C(═O)O— or a direct bond;
G¹ is Ci-C₂ alkylene, —(C═O)—, —O(C═O)—, —SC(═O)—, —R^{a}C(═O)— or a direct bond:
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁵ and R⁶ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In some embodiments, the lipid nanoparticle comprises a lipid having the structure:

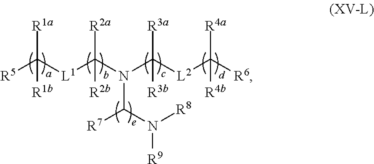

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent RID and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4b} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2,
provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, the lipid nanoparticle comprises a lipid having the structure:

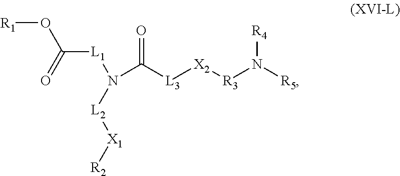

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

In some embodiments, the lipid nanoparticle comprises an ionizable lipid having the structure:

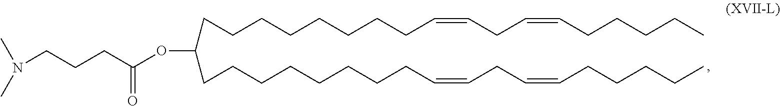 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, the lipid nanoparticle comprises a lipid having the structure:

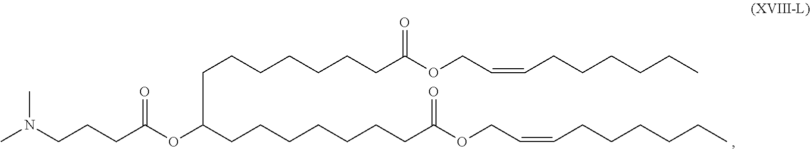 [SMILES: CCCCCC/C=C\COC(=O)CCCCCCCC(CCCCCCCC(=O)OC/C=C\CCCCCC)OC(=O)CCCN(C)C]

or a pharmaceutically acceptable salt thereof.

In some embodiments, the lipid nanoparticle comprises a lipid having the structure:

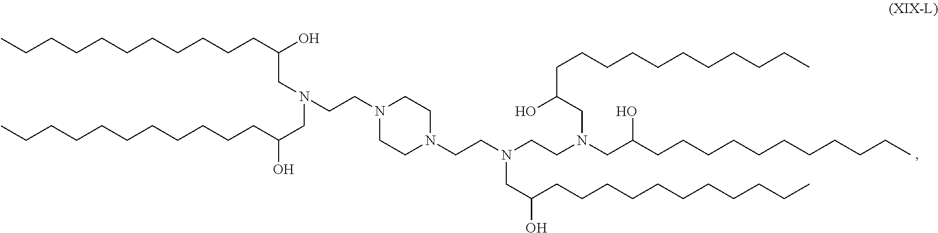 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, the lipid nanoparticle comprises a lipid having the structure:

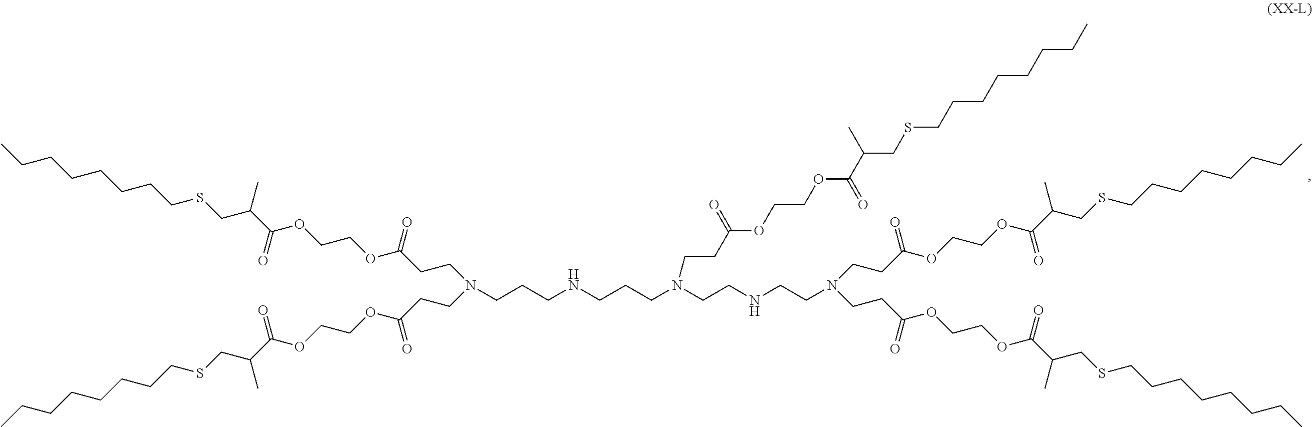 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, the lipid nanoparticle comprises a lipid having the structure:

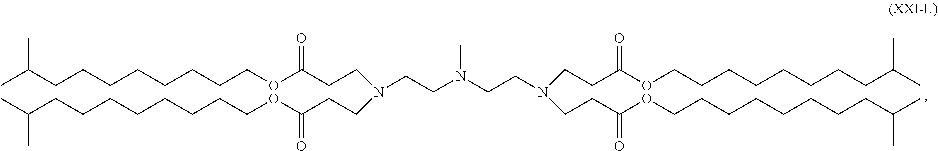 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, the lipid nanoparticle comprises a lipid having the structure:

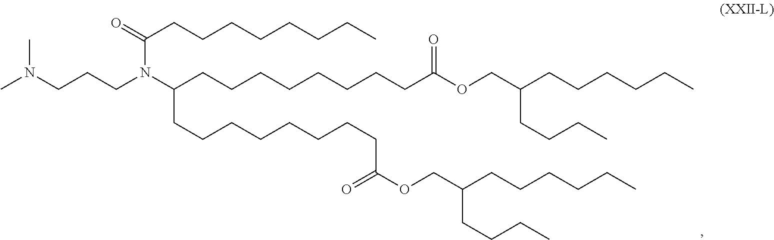 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, the lipid nanoparticle comprises a lipid having the structure:

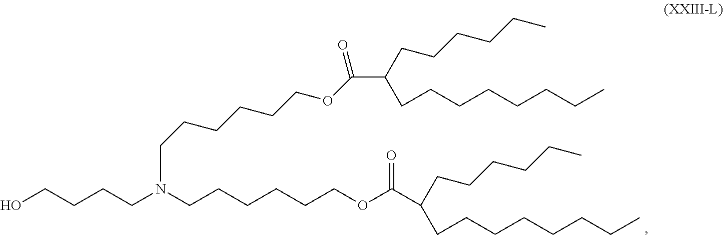 [SMILES: CCCCCCCCC(CCCCCC)C(=O)OCCCCCCN(CCCCO)CCCCCCOC(=O)C(CCCCCC)CCCCCCCC]

or a pharmaceutically acceptable salt thereof.

In some embodiments, the lipid nanoparticle comprises a lipid having the structure:

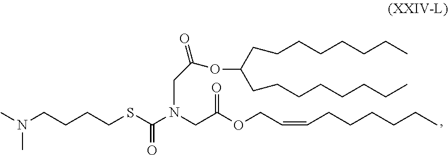 [SMILES: CCCCCC/C=C\COC(=O)CN(CC(=O)OC(CCCCCCCC)CCCCCCCC)C(=O)SCCCCN(C)C]

or a pharmaceutically acceptable salt thereof.

In some embodiments, the lipid nanoparticle comprises a lipid having the structure:

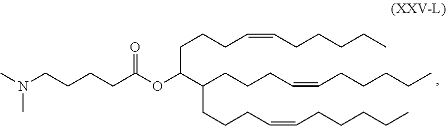 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, the lipid nanoparticle comprises a lipid having the structure:

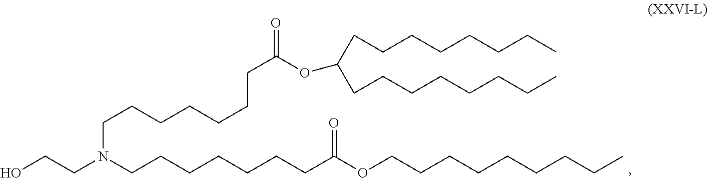 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, the lipid nanoparticle comprises a lipid having the structure:

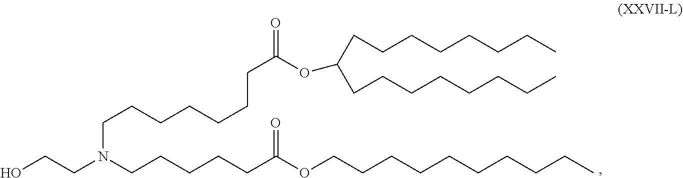 [SMILES: CCCCCCCCCCOC(=O)CCCCCN(CCO)CCCCCCCC(=O)OC(CCCCCCCC)CCCCCCCC]

or a pharmaceutically acceptable salt thereof.

### Non-Cationic Lipids

In certain embodiments, the lipid nanoparticles described herein comprise one or more non-cationic lipids. Non-cationic lipids may be phospholipids.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, the lipid nanoparticle comprises 5-15 mol %, 5-10 mol %, or 10-15 mol % DSPC. For example, the lipid nanoparticle may comprise 5, 6, 7, 8, 9, 10, 11, 12, 13, 14, or 15 mol % DSPC.

In certain embodiments, the lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidyl glycerols, and phosphatidic acids. Phospholipids also include phosphosphingolipid, such as sphingomyelin.

In some embodiments, a phospholipid comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid is an analog or variant of DSPC. In certain embodiments, a phospholipid useful or potentially is a compound of Formula (IX):

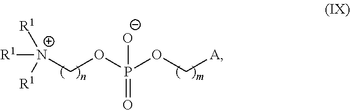

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

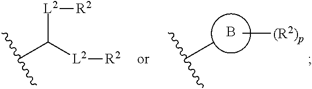

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group,
Ring B is optionally substituted carbocyclyl, optionally substituted heterocyclyl, optionally substituted aryl, or optionally substituted heteroaryl; and
p is 1 or 2;
provided that the compound is not of the formula:

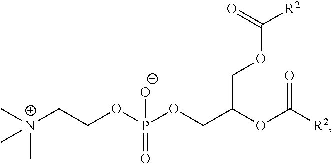

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, the lipid nanoparticle comprises a molar ratio of 5-25% non-cationic lipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% non-cationic lipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 5-25% phospholipid relative to the other lipid components. For example, the lipid nanoparticle may comprise a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, the lipid nanoparticle comprises a molar ratio of 25-55% structural lipid relative to the other lipid components. For example, the lipid nanoparticle may comprise a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises 30-45 mol % sterol, optionally 35-40 mol %, for example, 30-31 mol %, 31-32 mol %, 32-33 mol %, 33-34 mol %, 35-35 mol %, 35-36 mol %, 36-37 mol %, 38-38 mol %, 38-39 mol %, or 39-40 mol %. In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 35-40 mol % cholesterol. For example, the lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-distearyl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

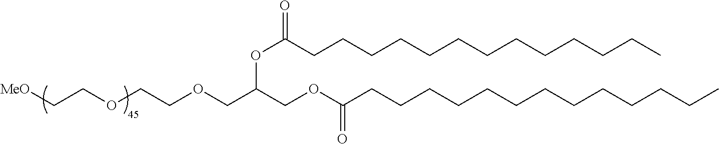

In some embodiments, PEG lipids can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment.

In certain embodiments, a PEG lipid is a compound of Formula (X):

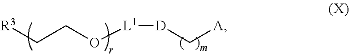

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S. C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

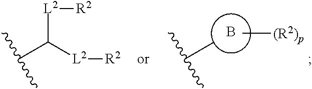

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S. C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)¿N(R^{N}), OS(O)₂N(R^{N}), or —N(R^{N})S(O)₂O;
each instance of R^{N} is independently hydrogen, optionally substituted alkyl, or a nitrogen protecting group,
Ring B is optionally substituted carbocyclyl, optionally substituted heterocyclyl, optionally substituted aryl, or optionally substituted heteroaryl; and
p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X—OH):

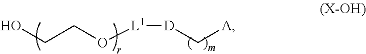

or a salt thereof.

In certain embodiments, a PEG lipid is a PEGylated fatty acid. In certain embodiments, a PEG lipid is a compound of Formula (XI). Described herein are compounds of Formula (XI):

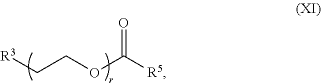

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and each instance of R^{N} is independently hydrogen, optionally substituted alkyl, or a nitrogen protecting group.

In certain embodiments, the compound of Formula (XI) is of Formula (XI-OH):

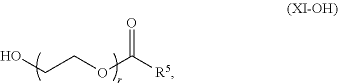

or a salt thereof. In some embodiments, r is 40-50.

In yet other embodiments the compound of Formula (XI) is:

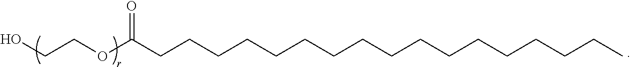

or a salt thereof.

In some embodiments, the compound of Formula (XI) is

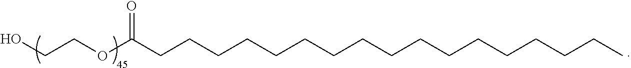

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, the lipid nanoparticle comprises a molar ratio of 0.5-15% PEG lipid relative to the other lipid components. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises 1-5% PEG-modified lipid, optionally 1-3 mol %, for example 1.5 to 2.5 mol %, 1-2 mol %, 2-3 mol %, 3-4 mol %, or 4-5 mol %. In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). Without being bound by theory, it is believed that spiking a LNP composition with additional PEG can provide benefits during lyophilization. Thus, some embodiments, comprise adding additional PEG as compared to an amount used for a non-lyophilized LNP composition. In embodiments comprise adding about 0.5 mol % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, the lipid nanoparticle comprises 20-60 mol % ionizable amino lipid, 5-25 mol % non-cationic lipid, 25-55 mol % sterol, and 0.5-15 mol % PEG-modified lipid.

In some embodiments, a LNP comprises an ionizable amino lipid of Compound 1, wherein the non-cationic lipid is DSPC, the structural lipid that is cholesterol, and the PEG lipid is DMG-PEG.

In some embodiments, a LNP comprises an ionizable amino lipid of any of Formula VI, VII or VIIII, a phospholipid comprising DSPC, a structural lipid, and a PEG lipid comprising PEG-DMG.

In some embodiments, a LNP comprises an ionizable amino lipid of any of Formula VI, VII or VIII, a phospholipid comprising DSPC, a structural lipid, and a PEG lipid comprising a compound having Formula XI.

In some embodiments, a LNP comprises an ionizable amino lipid of Formula VI, VII or VIII, a phospholipid comprising a compound having Formula VIII, a structural lipid, and the PEG lipid comprising a compound having Formula X or XI.

In some embodiments, a LNP comprises an ionizable amino lipid of Formula VI, VII or VIII, a phospholipid comprising a compound having Formula IX, a structural lipid, and the PEG lipid comprising a compound having Formula X or XI.

In some embodiments, a LNP comprises an ionizable amino lipid of Formula VI, VII or VIII, a phospholipid having Formula IX, a structural lipid, and a PEG lipid comprising a compound having Formula XI.

In some embodiments, the lipid nanoparticle comprises 49 mol % ionizable amino lipid, mol % DSPC, 38.5 mol % cholesterol, and 2.5 mol % DMG-PEG In some embodiments, the lipid nanoparticle comprises 49 mol % ionizable amino lipid, 11 mol % DSPC, 38.5 mol % cholesterol, and 1.5 mol % DMG-PEG.

In some embodiments, the lipid nanoparticle comprises 48 mol % ionizable amino lipid, 11 mol % DSPC, 38.5 mol % cholesterol, and 2.5 mol % DMG-PEG.

In some embodiments, a LNP comprises an N:P ratio of from about 2:1 to about 30:1.

In some embodiments, a LNP comprises an N:P ratio of about 6:1.

In some embodiments, a LNP comprises an N:P ratio of about 3:1, 4:1, or 5:1.

In some embodiments, a LNP comprises a wt/wt ratio of the ionizable amino lipid component to the RNA of from about 10:1 to about 100:1.

In some embodiments, a LNP comprises a wt/wt ratio of the ionizable amino lipid component to the RNA of about 20:1.

In some embodiments, a LNP comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

A LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, a LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprises an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising the lipid nanoparticles, such as those described herein, may be used for a wide variety of applications, including the stealth delivery of therapeutic payloads with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, the lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, a LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

In some embodiments, a subject to which a composition comprising a nucleic acid and a lipid, is administered is a subject that suffers from or is at risk of suffering from a disease, disorder or condition, including a communicable or non-communicable disease, disorder or condition. As used herein, “treating” a subject can include either therapeutic use or prophylactic use relating to a disease, disorder or condition, and may be used to describe uses for the alleviation of symptoms of a disease, disorder or condition, uses for vaccination against a disease, disorder or condition, and uses for decreasing the contagiousness of a disease, disorder or condition, among other uses.

In some embodiments the nucleic acid is an mRNA vaccine designed to achieve particular biologic effects. Exemplary vaccines feature mRNAs encoding a particular antigen of interest (or an mRNA or mRNAs encoding antigens of interest). In exemplary aspects, the vaccines feature an mRNA or mRNAs encoding antigen(s) derived from infectious diseases or cancers.

Diseases or conditions, in some embodiments include those caused by or associated with infectious agents, such as bacteria, viruses, fungi and parasites. Non-limiting examples of such infectious agents include Gram-negative bacteria, Gram-positive bacteria, RNA viruses (including (+)ssRNA viruses, (−)ssRNA viruses, dsRNA viruses), DNA viruses (including dsDNA viruses and ssDNA viruses), reverse transcriptase viruses (including ssRNA-RT viruses and dsDNA-RT viruses), protozoa, helminths, and ectoparasites.

Thus, the disclosure also encompasses infectious disease vaccines. The antigen of the infectious disease vaccine is a viral or bacterial antigen.

In some embodiments, a disease, disorder, or condition is caused by or associated with a virus.

Some embodiments of lyophilized compositions are also useful for treating or preventing a symptom of diseases characterized by missing or aberrant protein activity, by replacing the missing protein activity or overcoming the aberrant protein activity. Because of the rapid initiation of protein production following introduction of mRNAs, as compared to viral DNA vectors, the compounds of the present disclosure are particularly advantageous in treating acute diseases such as sepsis, stroke, and myocardial infarction. Moreover, the lack of transcriptional regulation of the alternative mRNAs of the present disclosure is advantageous in that accurate titration of protein production is achievable. Multiple diseases are characterized by missing (or substantially diminished such that proper protein function does not occur) protein activity. Such proteins may not be present, are present in very low quantities or are essentially non-functional. In some embodiments, the disclosure relates to a method for treating such conditions or diseases in a subject by introducing polynucleotide or cell-based therapeutics containing the alternative polynucleotides described herein, wherein the alternative polynucleotides encode for a protein that replaces the protein activity missing from the target cells of the subject.

Diseases characterized by dysfunctional or aberrant protein activity include, but are not limited to, cancer and other proliferative diseases, genetic diseases (e.g., cystic fibrosis), autoimmune diseases, diabetes, neurodegenerative diseases, cardiovascular diseases, and metabolic diseases. In some embodiments, the disclosure relates to a method for treating such conditions or diseases in a subject by introducing polynucleotide or cell-based therapeutics containing the polynucleotides, wherein the polynucleotides encode for a protein that antagonizes or otherwise overcomes the aberrant protein activity present in the cell of the subject.

In some embodiments, a composition disclosed herein does not comprise a pharmaceutical preservative. In other embodiments, a composition disclosed herein does comprise a pharmaceutical preservative. Non-limiting examples of pharmaceutical preservatives include methyl paragen, ethyl paraben, propyl paraben, butyl paraben, benzyl alcohol, chlorobutanol, phenol, meta cresol (m-cresol), chloro cresol, benzoic acid, sorbic acid, thiomersal, phenylmercuric nitrate, bronopol, propylene glycol, benzalkonium chloride, and benzethionium chloride. In some embodiments, a composition disclosed herein does not comprise phenol, m-cresol, or benzyl alcohol Compositions in which microbial growth is inhibited may be useful in the preparation of injectable formulations, including those intended for dispensing from multi-dose vials. Multi-dose vials refer to containers of pharmaceutical compositions from which multiple doses can be taken repeatedly from the same container. Compositions intended for dispensing from multi-dose vials typically must meet USP requirements for antimicrobial effectiveness.

In some embodiments, “administering” or “administration” means providing a material to a subject in a manner that is pharmacologically useful. In some embodiments, a composition disclosed herein is administered to a subject enterally. In some embodiments, an enteral administration of the composition is oral. In some embodiments, a composition disclosed herein is administered to the subject parenterally. In some embodiments, a composition disclosed herein is administered to a subject subcutaneously, intraocularly, intravitreally, subretinally, intravenously (IV), intracerebro-ventricularly, intramuscularly, intrathecally (IT), intracisternally, intraperitoneally, via inhalation, topically, or by direct injection to one or more cells, tissues, or organs.

To “treat” a disease as the term is used herein, means to reduce the frequency or severity of at least one sign or symptom of a disease, disorder or condition experienced by a subject. The compositions described above or elsewhere herein are typically administered to a subject in an effective amount, that is, an amount capable of producing a desirable result. The desirable result will depend upon the active agent being administered. For example, an effective amount of a composition comprising a nucleic acid and a lipid may be an amount of the composition that is capable of increasing expression of a protein in the subject. A therapeutically acceptable amount may be an amount that is capable of treating a disease or condition, e.g., a disease or condition that that can be relieved by increasing expression of a protein in a subject. As is well known in the medical and veterinary arts, dosage for any one subject depends on many factors, including the subject's size, body surface area, age, the particular composition to be administered, the active ingredient(s) in the composition, the intended outcome of the administration, time and route of administration, general health, and other drugs being administered concurrently.

In some embodiments, a subject is administered a composition comprising a nucleic acid and a lipid I in an amount sufficient to increase expression of a protein in the subject.

In certain embodiments, LNP preparations (e.g., populations or formulations) are analyzed for polydispersity in size (e.g., particle diameter) and/or composition (e.g., amino lipid amount or concentration, phospholipid amount or concentration, structural lipid amount or concentration, PEG-lipid amount or concentration, mRNA amount (e.g., mass) or concentration) and, optionally, further assayed for in vitro and/or in vivo activity. Fractions or pools thereof can also be analyzed for accessible mRNA and/or purity (e.g., purity as determined by reverse-phase (RP) chromatography).

Particle size (e.g., particle diameter) can be determined by Dynamic Light Scattering (DLS). DLS measures a hydrodynamic diameter. Smaller particles diffuse more quickly, leading to faster fluctuations in the scattering intensity and shorter decay times for the autocorrelation function. Larger particles diffuse more slowly, leading to slower fluctuations in the scattering intensity and longer decay times in the autocorrelation function.

mRNA purity can be determined by reverse phase high-performance liquid chromatography (RP-HPLC) size based separation. This method can be used to assess mRNA integrity by a length-based gradient RP separation and UV detection of RNA at 260 nm. As used herein “main peak” or “main peak purity” refers to the RP-HPLC signal detected from mRNA that corresponds to the full size mRNA molecule loaded within a given LNP formulation. mRNA purity can also be assessed by fragmentation analysis. Fragmentation analysis (FA) is a method by which nucleic acid (e.g., mRNA) fragments can be analyzed by capillary electrophoresis. Fragmentation analysis involves sizing and quantifying nucleic acids (e.g., mRNA), for example by using an intercalating dye coupled with an LED light source. Such analysis may be completed, for example, with a Fragment Analyzer from Advanced Analytical Technologies, Inc.

Compositions formed via the methods described herein may be particularly useful for administering an agent to a subject in need thereof. In some embodiments, the compositions are used to deliver a pharmaceutically active agent. In some instances, the compositions are used to deliver a prophylactic agent. The compositions may be administered in any way known in the art of drug delivery, for example, orally, parenterally, intravenously, intramuscularly, subcutaneously, intradermally, transdermally, intrathecally, submucosally, etc.

Once the compositions have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions described herein and for use in accordance with the embodiments described herein may include a pharmaceutically acceptable excipient. As used herein, the term “pharmaceutically acceptable excipient” means a non-toxic, inert solid, semi-solid or liquid filler, diluent, encapsulating material or formulation auxiliary of any type Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions can be administered to humans and/or to animals, orally, parenterally, intracisternally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragées, capsules, pills, and granules can be prepared with coatings and shells such as enteric coatings and other coatings well known in the pharmaceutical formulating art They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also possible.

The ointments, pastes, creams, and gels may contain, in addition to the compositions, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the compositions, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compositions in a proper medium. Absorption enhancers can also be used to increase the flux of the compound across the skin. The rate can be controlled by either providing a rate controlling membrane or by dispersing the compositions in a polymer matrix or gel.

In other embodiments, the compositions are loaded and stored in prefilled syringes and cartridges for patient-friendly autoinjector and infusion pump devices.

Some embodiments relate to kits for use in preparing or administering the compositions described herein. A kit for forming compositions may include any solvents, solutions, buffer agents, acids, bases, salts, targeting agent, etc. needed in the composition formation process. Different kits may be available for different targeting agents. In certain embodiments, the kit includes materials or reagents for purifying, sizing, and/or characterizing the resulting compositions. The kit may also include instructions on how to use the materials in the kit. The one or more agents (e.g., pharmaceutically active agent) to be contained within the composition are typically provided by the user of the kit.

Kits for using or administering the compositions are also described herein. The compositions may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the compositions. Exemplary devices include syringes, spoons, measuring devices, etc. The kit may optionally include instructions for administering the compositions (e.g., prescribing information).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻, salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As disclosed herein, the terms “composition” and “formulation” are used interchangeably.

### Nucleic Acids

Some aspects of the present disclosure relate to compositions and methods of preparing lyophilized compositions comprising a nucleic acid formulated in a lipid nanoparticle. In some embodiments, the nucleic acid is an mRNA. The nucleic acids, for example mRNAs, are preferably formulated in appropriate carriers or delivery vehicles (e.g., lipid nanoparticles), such that the nucleic acids, e.g., mRNAs, are suitable for use in vivo. When appropriately formulated, nucleic acids, e.g., mRNAs, are capable of being delivered to cells and/or tissues within a subject, e.g., a human subject, to effectuate translation of protein encoded by these nucleic acids. As used herein, the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G))). As used herein, the term nucleic acid refers to polyribonucleotides as well as polydeoxyribonucleotides. The term nucleic acid shall also include polynucleosides (i.e., a polynucleotide minus the phosphate) and any other organic base containing polymer. Non-limiting examples of nucleic acids include chromosomes, genomic loci, genes or gene segments that encode polynucleotides or polypeptides, coding sequences, non-coding sequences (e.g., intron, 5′-UTR, or 3′-UTR) of a gene, pri-mRNA, pre-mRNA, cDNA, mRNA, etc. A nucleic acid (e.g., mRNA) may include a substitution and/or modification. In some embodiments, the substitution and/or modification is in one or more bases and/or sugars. For example, in some embodiments a nucleic acid (e.g., mRNA) includes nucleic acids having backbone sugars that are covalently attached to low molecular weight organic groups other than a hydroxyl group at the 2′ position and other than a phosphate group or hydroxy group at the 5′ position. Thus, in some embodiments, a substituted or modified nucleic acid (e.g., mRNA) includes a 2′-O-alkylated ribose group. In some embodiments, a modified nucleic acid (e.g., mRNA) includes sugars such as hexose, 2′-F hexose, 2′-amino ribose, constrained ethyl (cEt), locked nucleic acid (LNA), arabinose or 2′-fluoroarabinose instead of ribose. Thus, in some embodiments, a nucleic acid (e.g., mRNA) is heterogeneous in backbone composition thereby containing any possible combination of polymer units linked together such as peptide-nucleic acids (which have an amino acid backbone with nucleic acid bases).

The nucleic acid sequences include nucleic acid sequences that have been removed from their naturally occurring environment, recombinant or cloned DNA isolates, and chemically synthesized analogues or analogues biologically synthesized by heterologous systems.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing. A nucleic may comprise naturally occurring nucleotides and/or non-naturally occurring nucleotides such as modified nucleotides.

When applied to a nucleic acid sequence, the term “isolated” in this context denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

A nucleic acid vector may include an insert which may be an expression cassette or open reading frame (ORF). An “open reading frame” is a continuous stretch of DNA beginning with a start codon (e.g., methionine (ATG)), and ending with a stop codon (e.g., TAA, TAG or TGA) and encodes a protein or peptide (e.g., a therapeutic protein or therapeutic peptide). In some embodiments, an expression cassette encodes a RNA including at least the following elements: a 5′ untranslated region, an open reading frame region encoding the mRNA, a 3′ untranslated region and a poly A tail. The open reading frame may encode any mRNA sequence, or portion thereof.

In some embodiments, a nucleic acid vector comprises a 5′ untranslated region (UTR). A “5′ untranslated region (UTR)” refers to a region of an mRNA that is directly upstream (i.e., 5′) from the start codon (i.e., the first codon of an mRNA transcript translated by a ribosome) that does not encode a protein or peptide.

In some embodiments, a nucleic acid vector comprises a 3′ untranslated region (UTR). A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

The terms 5′ and 3′ are used herein to describe features of a nucleic acid sequence related to either the position of genetic elements and/or the direction of events (5′ to 3′), such as e.g. transcription by RNA polymerase or translation by the ribosome which proceeds in 5′ to 3′ direction. Synonyms are upstream (5′) and downstream (3′). Conventionally, DNA sequences, gene maps, vector cards and RNA sequences are drawn with 5′ to 3′ from left to right or the 5′ to 3′ direction is indicated with arrows, wherein the arrowhead points in the 3′ direction. Accordingly, 5′ (upstream) indicates genetic elements positioned towards the left-hand side, and 3′ (downstream) indicates genetic elements positioned towards the right-hand side, when following this convention.

A nucleic acid (e.g., mRNA) typically comprises a plurality of nucleotides. A nucleotide includes a nitrogenous base, a five-carbon sugar (ribose or deoxyribose), and at least one phosphate group. Nucleotides include nucleoside monophosphates, nucleoside diphosphates, and nucleoside triphosphates. A nucleoside monophosphate (NMP) includes a nucleobase linked to a ribose and a single phosphate; a nucleoside diphosphate (NDP) includes a nucleobase linked to a ribose and two phosphates; and a nucleoside triphosphate (NTP) includes a nucleobase linked to a ribose and three phosphates. Nucleotide analogs are compounds that have the general structure of a nucleotide or are structurally similar to a nucleotide. Nucleotide analogs, for example, include an analog of the nucleobase, an analog of the sugar and/or an analog of the phosphate group(s) of a nucleotide.

A nucleoside includes a nitrogenous base and a 5-carbon sugar. Thus, a nucleoside plus a phosphate group yields a nucleotide. Nucleoside analogs are compounds that have the general structure of a nucleoside or are structurally similar to a nucleoside. Nucleoside analogs, for example, include an analog of the nucleobase and/or an analog of the sugar of a nucleoside.

It should be understood that the term “nucleotide” includes naturally-occurring nucleotides, synthetic nucleotides and modified nucleotides, unless indicated otherwise. Non-limiting examples of naturally-occurring nucleotides used for the production of RNA, e.g., in an IVT reaction, include adenosine triphosphate (ATP), guanosine triphosphate (GTP), cytidine triphosphate (CTP), uridine triphosphate (UTP), and 5-methyluridine triphosphate (mSUTP). In some embodiments, adenosine diphosphate (ADP), guanosine diphosphate (GDP), cytidine diphosphate (CDP), and/or uridine diphosphate (UDP) are used.

Examples of nucleotide analogs include, but are not limited to, antiviral nucleotide analogs, phosphate analogs (soluble or immobilized, hydrolyzable or non-hydrolyzable), dinucleotide, trinucleotide, tetranucleotide, e.g., a cap analog, or a precursor/substrate for enzymatic capping (vaccinia or ligase), a nucleotide labeled with a functional group to facilitate ligation/conjugation of cap or 5′ moiety (IRES), a nucleotide labeled with a 5′ PO4 to facilitate ligation of cap or 5′ moiety, or a nucleotide labeled with a functional group/protecting group that can be chemically or enzymatically cleaved. Examples of antiviral nucleotide/nucleoside analogs include, but are not limited, to Ganciclovir, Entecavir, Telbivudine, Vidarabine and Cidofovir.

Modified nucleotides may include modified nucleobases. For example, a RNA transcript (e.g., mRNA transcript) of the present disclosure may include a modified nucleobase selected from pseudouridine (ψ), 1-methylpseudouridine (m1ψ), 1-ethylpseudouridine, 2-thiouridine, 4′-thiouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine (mo5U) and 2′-O-methyl uridine. In some embodiments, a RNA transcript (e.g., mRNA transcript) includes a combination of at least two (e.g., 2, 3, 4 or more) of the foregoing modified nucleobases.

Some embodiments comprise compositions with at least about 0.25 mg/mL nucleic acid (e.g., mRNA), such as 0.5 mg/mL, 0.75 mg/mL, 1 mg/mL, 1.25 mg/mL, 1.5 mg/mL, or 2 mg/mL nucleic acid.

In some embodiments, the nucleic acid is not self-amplifying. Self-amplifying nucleic acids (e.g., self-amplifying RNAs) encode proteins such as viral replicases that are capable of using the nucleic acid encoding the replicase as a template for replication, allowing copying of the self-amplifying nucleic acid within a cell. Because a self-amplifying nucleic acid may be replicated in cell to which it is introduced, a given dose of the self-amplifying nucleic acid may lead to production of more of an encoded non-replicase protein (e.g., antigen) than an equivalent dose of a nucleic acid that encodes the same protein but is not self-amplifying (e.g., an mRNA).

In some embodiments, the nucleic acids (e.g., mRNAs) of the methods and compositions encode an immunogenic protein, which may be translated in vivo from the nucleic acid following administration of the composition to a subject, e.g., a human. Translation of an immunogenic protein in vivo elicits an immune response targeting the immunogenic protein. Immune responses targeting a protein may include the generation of antibodies that specifically bind to and/or neutralize the protein, the generation of B cells encoding antibodies specific to the protein, and the generation of T cells with receptors that bind peptides with sequences present in the sequence of the protein.

The entire contents of International Application Nos. PCT/US2015/027400, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

### Lyophilized Compositions

In some embodiments, the methods comprise obtaining lyophilized compositions with a moisture content below a certain value. As used herein, “moisture content” refers to the percentage (by weight) of a composition that is comprised of water. Methods of measuring moisture content of a lyophilized composition include loss-on-drying (LOD) analysis, thermogravimetric (TGA) analysis, and Karl Fischer titration (see, e.g., FDA, Docket No. 89D-0140, Guideline for the Determination of Residual Moisture in Dried Biological Products (Center for Biologics Evaluation and Research, January 1990)).

In some embodiments, the lyophilized composition has a moisture content of 6.0% w/w or less, 5.0% w/w or less, 4.0% w/w or less, 3.5% w/w or less, 3.0% w/w or less, 2.5% w/w or less, 2.0% w/w or less, 1.5% w/w or less, 1% w/w or less, 0.75% w/w or less, 0.5% w/w or less, or 0.25% w/w or less. In some embodiments, the lyophilized composition has a moisture content of 3.0% w/w or less.

In some embodiments, the methods comprise obtaining lyophilized compositions in which the coefficient of degradation of a nucleic acid in the composition is below a predetermined value. As used herein, a “coefficient of degradation” refers to a parameter of an equation describing the loss of nucleic acid purity over time. As used herein, “nucleic acid purity” refers to the percentage of nucleic acid in a composition having a desired sequence and structure. Compositions of the present disclosure are prepared using nucleic acids having a specific sequence encoding a protein to be expressed in cells. During the course of lyophilization and/or storage after lyophilization, the nucleic acid may be degraded by environmental factors such as water or nucleases. Water molecules can hydrolyze the phosphodiester bond that bridges a phosphate moiety and sugar moiety in the sugar-phosphate backbone of a nucleic acid, resulting in the production of two separate nucleic acid molecules, neither of which contains an intact sequence encoding the full-length protein encoded by the unhydrolyzed nucleic acid. Nucleases are enzymes that can facilitate this process, but nucleic acids are susceptible to degradation by water molecules even in the absence of environmental nucleases. Nucleic acid purity may be measured by any one of multiple methods known in the art, such as mass spectrometry or high-performance liquid chromatography (HPLC) (see, e.g., Papadoyannis et al. J Liq Chrom Relat Tech. 2007. 27(6): 1083-1092). In HPLC, a sample to be analyzed, such as nucleic acid, is dissolved in a solvent (mobile phase) and passed through a column containing a solid material (stationary phase), with a detector measuring the presence of dissolved sample molecules as the mobile phase is eluted from the column. The rate at which molecules of the sample move through the stationary phase depends on multiple factors, including size, such that different components of the sample will be observed at different times. A sample containing 100% pure nucleic acid will produce a single peak (main peak) on a chromatogram when analyzed by HPLC, while a sample containing multiple different nucleic acid molecules will produce multiple peaks, including a main peak and one or more impurity peaks, for a total of N peaks. To calculate the purity of a nucleic acid using HPLC analysis, the area under the curve (A.U.C.) of each of N peaks is calculated by integration, and the percent purity is calculated using the equation %

$$\% purity = \frac{A U C ( main peak )}{{\sum }_{i = 1}^{N} A U C ( {peak}_{i} )} .$$

Loss of nucleic acid purity over time may be described by a differential equation of the form

$$\frac{dP}{dt} = - λ P ,$$

where P is nucleic acid purity (%), λ is the coefficient of degradation, and dP/dt is the rate of change in nucleic acid purity. Alternatively, nucleic acid purity over time may be described by an equation of the form P(t)=P₀ₑ^{-λt}, where P(t) is nucleic acid purity (%) at a given time, t, P₀ is initial nucleic acid purity at time t=0, e is the base of the natural logarithm, and λ is the coefficient of degradation. In both equation forms, a positive value of λ indicates exponential decay, while a negative λ indicates exponential growth, with larger absolute values of λ indicating faster decay or growth, respectively. In some embodiments, the coefficient of degradation is expressed in units of month⁻¹. In some embodiments, the nucleic acid of the lyophilized composition has a coefficient of degradation at 5° C. of 0.05 month⁻¹ or less, 0.04 month⁻¹ or less, 0.03 month⁻¹ or less, or 0.02 month⁻¹ or less. In some embodiments, the coefficient of degradation is 0.02 month⁻¹ or less. In some embodiments, the coefficient of degradation is 0.01 month⁻¹ or less. In some embodiments, the coefficient of degradation is 0.01 month⁻¹ or less, 0.008 month⁻¹ or less, 0.006 month⁻¹ or less, or 0.004 month⁻¹ or less. In some embodiments, the coefficient of degradation is 0.004 month⁻¹ or less.

In some embodiments, the nucleic acid in a lyophilized LNP degrades (e.g., as measured by capillary electrophoresis) about 2% or less per month during storage, such as about 1% or less, about 0.75% or less, about 0.5% or less, about 0.4% or less, about 0.3% or less, about 0.2% or less, or about 0.1% or less per month during storage (e.g., at 5° C.). In some embodiments, the methods comprise obtaining lyophilized compositions in which the nucleic acid in the lyophilized composition is at least 50% pure (such as about 50% pure, about 55% pure, about 60% pure, about 65% pure, about 70% pure, or about 75% pure or more) after storage at 0° C. or more (such as 0° C., 2° C., 5° C., 8° C., 10° C., 15° C., 20° C., 25° C., or 2-8° C.) for a given length of time. The length of time for which a composition will comprise at least 50% pure nucleic acid can reliably be predicted by measuring a) the initial purity of the nucleic acid in a composition, and b) the coefficient of degradation of nucleic acid, as described above, then using the equation P(t)=P₀ₑ^{λt} to calculate the value of t at which P(t)=50% or 0.5. This length of time is given by the formula

$$t = \frac{ln 50 \% - ln {P}_{0}}{- λ}$$

if P₀ is expressed as a percentage or

$$t = \frac{ln 0.5 - ln {P}_{0}}{- λ}$$

if P₀ is expressed as a proportion.

In some embodiments, the nucleic acid in the lyophilized composition is at least 50% pure (such as about 50% pure, about 55% pure, about 60% pure, about 65% pure, about 70% pure, or about 75% pure or more) after at least 12 months, at least 18 months, at least 21 months, at least 24 months, at least 27 months, at least 30 months, at least 33 months, or at least 36 months of storage. In some embodiments, the nucleic acid in the lyophilized composition is at least 50% pure (such as about 50% pure, about 55% pure, about 60% pure, about 65% pure, about 70% pure, or about 75% pure or more) after least 24 months of storage. In some 2.5 embodiments, the storage is conducted at a temperature between about 2° C. and about 8° C. In some embodiments, the storage is conducted at about 5° C. Degradation of nucleic acids is a chemical reaction that occurs more readily at higher temperatures, and as such the coefficient of degradation depends on the temperature at which nucleic acids are stored.

In some embodiments, substantially no cholesterol crystals (e.g., no cholesterol crystals) can be detected in the formulation following 6 months or more of storage (such as after 6 months, 9 months, 12 months, 18 months, or 24 months or more of storage).

Some aspects of the disclosure relate to a lyophilized composition produced by any of the methods provided herein. In some embodiments, the composition is in a solid form.

Some aspects of the disclosure relate to lyophilized pharmaceutical compositions comprising an mRNA, a lyoprotectant and a lipid nanoparticle, wherein the lipid nanoparticle comprises ionizable amino lipid; non-cationic lipid; sterol; and PEG-modified lipid, wherein the composition has a moisture content of less than or equal to 6.0% w/w.

Some aspects of the disclosure relate to lyophilized pharmaceutical compositions comprising an mRNA, a lyoprotectant and a lipid nanoparticle, wherein the lipid nanoparticle comprises ionizable amino lipid; non-cationic lipid; sterol; and PEG-modified lipid, wherein the ratio of lyoprotectant to lipid molecules is about 8:1 to about 40:1.

Some aspects of the disclosure relate to lyophilized pharmaceutical compositions comprising an mRNA, a lyoprotectant and a lipid nanoparticle, wherein the lipid nanoparticle comprises ionizable amino lipid; non-cationic lipid; sterol; and PEG-modified lipid, wherein the PEG-modified lipid is a C18 stearic-OH-PEG, and wherein the composition is stable for at least about 9 months upon storage at about 4° C.

In some embodiments of the lyophilized compositions, the composition has a moisture content of less than or equal to 6.0% w/w.

In some embodiments of the lyophilized compositions, the PEG-modified lipid is a C18 stearic-OH-PEG. In some embodiments, the composition is stable for at least about 9 months upon storage at about 4° C.

In some embodiments of the lyophilized compositions, the ratio of lyoprotectant to lipid molecules is about 8:1 to about 40:1. In some embodiments, the ratio of lyoprotectant to lipid molecules is about 8:1 to about 20:1. In some embodiments, the ratio of lyoprotectant to lipid molecules is about 8:1, about 9:1, about 10:1, about 11:1, or about 12:1. In some embodiments, the ratio of sugar molecules to lipid molecules is at least 8:1.

In some embodiments, the PEG-modified lipid is:

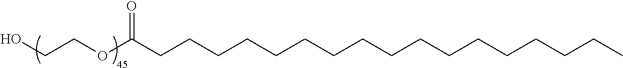

In some embodiments, the ionizable amino lipid is:

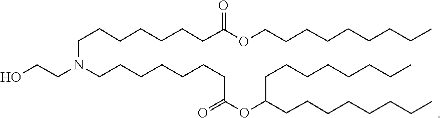 [SMILES: CCCCCCCCCOC(=O)CCCCCCCN(CCO)CCCCCCCC(=O)OC(CCCCCCCC)CCCCCCCC]

In some embodiments, the lyophilized composition does not comprise protamine. Previous formulations for RNA vaccine delivery included protamine complexed with RNA, and taught that such complexation of RNA with protamine was necessary to achieve sustained protective immune responses observed in experiments. See, e.g., Petsch et al., Nat Biotechnol. 2012. 30(12): 1210-1218. By contrast, some embodiments of lyophilized compositions comprising a lipid nanoparticle and mRNA are capable of eliciting an immune response to encoded proteins without the use of protamine for mRNA delivery, as shown in the Examples.

Some embodiments of lyophilized pharmaceutical compositions and/or compositions made by lyophilization methods comprise low moisture contents. Without wishing to be bound by theory, it is believed that reduced availability of water molecules in a lyophilized composition reduce the frequency of hydrolysis of nucleic acids (e.g., mRNAs) in a lyophilized composition, thereby improving stability of the nucleic acids in the composition, such as during extended storage over several months. Moisture contents may be measured by any method known in the art, such as Karl Fischer (KF) titration, thermogravimetry (TG), and/or gas chromatography. See, e.g., Roggo et al., J Pharm Biomed Anal. 2007. 44(3): 689-700; Blanco et al., J Pharm Biomed Anal. 1997. 16(2): 255-262; Zhou et al., J Pharm Sci. 2003. 92(5): 1058-1065. In some embodiments, the composition has a moisture content of less than or equal to 6.0% w/w. In some embodiments, the composition has a moisture content of less than or equal to 5.0% w/w. In some embodiments, the composition has a moisture content of less than or equal to 4.0% w/w. In some embodiments, the composition has a moisture content of less than or equal to 3.5% w/w. In some embodiments, the composition has a moisture content of less than or equal to 3.0% w/w. In some embodiments, the composition has a moisture content of less than or equal to 2.5% w/w. In some embodiments, the composition has a moisture content of less than or equal to 2.0% w/w. In some embodiments, the composition has a moisture content of less than or equal to 1.0% w/w. In some embodiments, the composition has a moisture content of less than or equal to 0.5% w/w. In some embodiments, the composition has a moisture content between 0.01% and 6.0% w/w, between 0.01% and 5.0% w/w, between 0.01% and 4.0% w/w, between 0.01% and 3.0% w/w, between 0.01% and 2.0% w/w, or between 0.01% and 1.0% w/w. In some embodiments, the composition has a moisture content between 0.01% w/w and 2.0% w/w. In some embodiments, the composition has a moisture content between 0.01% w/w and 1.0% w/w. In some embodiments, the composition has a moisture content between 0.01% w/w and 0.5% w/w. In some embodiments, moisture content is measured within 1 month after the end of the desorption step. In some embodiments, moisture content refers to the amount of moisture in a lyophilized composition after 1 or more (e.g., 3, 6, 9, 12, 24, 36, 48, 60, 72, 84, 96, 108, 120, or more) months of storage.

In some embodiments, upon reconstitution the lipid nanoparticle has a diameter (or a composition has a mean lipid particle diameter) of 120 nm or less, such as 80 nm or less, 70 nm or less, 60 nm or less, 50 nm or less, 40 nm or less, 30 nm or less, or 20 nm or less. In some embodiments, upon reconstitution the lipid nanoparticle (or a composition has a mean lipid particle diameter) has a diameter of at most 30 nm. In some embodiments, the lipid nanoparticle has a diameter from 5-120 nm, 5-80 nm, 5-70 nm, 5-60 nm, 5-50 nm, 5-40 nm, 5-30 nm, or 5-20 nm.

In some embodiments, the lyophilization increases the lipid nanoparticle size (or a mean lipid particle diameter) (e.g., as determined by DLS) by about 30 nm or less, such as about 25 nm or less, about 20 nm or less, about 15 nm or less, about 10 nm or less, or about 5 nm or less. In some embodiments, the lyophilization does not measurably increase the lipid nanoparticle size (or mean lipid particle diameter).

In some embodiments, a coefficient of degradation at 5° C. of the mRNA in the lyophilized composition is at most 0.05 month⁻¹, at most 0.04 month⁻¹, at most 0.03 month⁻¹, at most 0.02 month⁻¹, or at most 0.01 month⁻¹. In some embodiments, the coefficient of degradation is at most 0.02 month⁻¹. In some embodiments, the coefficient of degradation is at most 0.01 month⁻¹. In some embodiments, the coefficient of degradation is between 0.0001 and 0.05 month⁻¹, 0.0001 and 0.04 month⁻¹, 0.0001 and 0.03 month⁻¹, 0.001 and 0.02 month⁻¹, or 0.001 and 0.01 month⁻¹. In some embodiments, the coefficient of degradation is between 0.0001 and 0.02 month⁻¹. In some embodiments, the coefficient of degradation is between 0.001 and 0.01 month⁻¹.

In some embodiments, the mRNA in the lyophilized composition is at least 50% pure after at least 12 months, at least 18 months, at least 21 months, at least 24 months, at least 27 months, at least 30 months, at least 33 months, or at least 36 months of storage. In some embodiments, the mRNA in the lyophilized composition is at least 50% pure after 12 to 120 months, 12 to 108 months, 12 to 96 months, 12 to 84 months, 12 to 72 months, 12 to 60 months, 12 to 54 months, 12 to 48 months, 12 to 42 months, 12 to 36 months, 12 to 30 months, 12 to 24 months, or 12 to 18 months of storage at a temperature between about 2° C. and about 8° C. In some embodiments, the storage is conducted at a temperature of about 4° C. In some embodiments, the storage is conducted at about 5° C.

In some embodiments, the mRNA in the lyophilized composition is at least 50% pure after at least 24 months of storage. In some embodiments, the mRNA in the lyophilized composition is at least 50% pure after 24 to 120 months, 24 to 108 months, 24 to 96 months, 24 to 84 months, 24 to 72 months, 24 to 60 months, 24 to 54 months, 24 to 48 months, 24 to 42 months, 24 to 36 months, or 24 to 30 months of storage at a temperature between about 2° C. and about 8° C. In some embodiments, the storage is conducted at a temperature of about 4° C. In some embodiments, the storage is conducted at a temperature between about 2° C. and about 8° C. In some embodiments, the storage is conducted at a temperature of about 4° C. In some embodiments, the storage is conducted at about 5° C.

In some embodiments, the moisture content of a lyophilized composition remains below 6.0% (w/w), 5.0%, 4.0%, 3.0%, 2.0%, 1.5%, 1.0%, or 0.5% after 3-12 months of storage. For example, in some embodiments, the moisture content of a composition after 12 months of storage at 2-8° C. is less than or equal to 6.0% w/w. In some embodiments, the moisture content of a composition after 3-12 months of storage at 2-8° C. is less than or equal to 5.0% w/w. In some embodiments, the moisture content of a composition after 3-12 months of storage at 2-8° C. is less than or equal to 4.0% w/w. In some embodiments, the moisture content of a composition after 3-12 months of storage at 2-8° C. is less than or equal to 3.5% w/w. In some embodiments, the moisture content of a composition after 3-12 months of storage at 2-8° C. is less than or equal to 3.0% w/w. In some embodiments, the moisture content of a composition after 3-12 months of storage at 2-8° C. is less than or equal to 2.5% w/w. In some embodiments, the moisture content of a composition after 3-12 months of storage at 2-8° C. is less than or equal to 2.0% w/w. In some embodiments, the moisture content of a composition after 3-12 months of storage at 2-8° C. is less than or equal to 2.0% w/w. In some embodiments, the moisture content of a composition after 3-12 months of storage at 2-8° C. is less than or equal to 1.5% w/w. In some embodiments, the moisture content of a composition after 3-12 months of storage at 2-8° C. is less than or equal to 1.0%. In some embodiments, the moisture content of a composition after 3-12 months of storage at 2-8° C. is less than or equal to 0.5%. In some embodiments, the moisture content of a lyophilized composition is less than or equal to 2.0% w/w after 3 or more, 4 or more, 5 or more, 6 or more, 9 or more, or 12 or more months of storage at 2-8° C.

In some embodiments, the moisture content of a lyophilized composition remains below 6.0% w/w after 3-12 months of storage at a given temperature and/or relative humidity. For example, in some embodiments, the moisture content of a composition after 3-12 months of storage at 20-30° C. is less than or equal to 6.0% w/w. In some embodiments, the moisture content of a composition after 3-12 months of storage at 20-30° C. is less than or equal to 5.0% w/w. In some embodiments, the moisture content of a composition after 3-12 months of storage at 20-30° C. is less than or equal to 4.0% w/w. In some embodiments, the moisture content of a composition after 3-12 months of storage at 20-30° C. is less than or equal to 3.5% w/w. In some embodiments, the moisture content of a composition after 3-12 months of storage at 20-30° C. is less than or equal to 3.0% w/w. In some embodiments, the moisture content of a composition after 3-12 months of storage at 20-30° C. is less than or equal to 2.5% w/w. In some embodiments, the moisture content of a composition after 3-12 months of storage at 20-30° C. is less than or equal to 2.0% w/w. In some embodiments, the moisture content of a composition after 3-12 months of storage at 20-30° C. is less than or equal to 2.0% w/w. In some embodiments, the moisture content of a composition after 3-12 months of storage at 20-30° C. is less than or equal to 1.5% w/w. In some embodiments, the moisture content of a composition after 3-12 months of storage at 20-30° C. is less than or equal to 1.0%. In some embodiments, the moisture content of a composition after 3-12 months of storage at 20-30° C. is less than or equal to 0.5%. In some embodiments, the moisture content of a lyophilized composition is less than or equal to 2.0% w/w after 3 or more, 4 or more, 5 or more, 6 or more, 9 or more, or 12 or more months of storage at 20-30° C. In some embodiments, the storage is conducted at 50-100%, 60-100%, 70-100%, 75%-100%, 80-100%, 90-100% or 95-100% relative humidity.

In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 6 months of storage. In some embodiments, the storage is conducted at a temperature between 2-8° C. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 9 months of storage. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 12 months of storage. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 24 months of storage. In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 36 months of storage. In some embodiments, the storage is conducted at a temperature between 10-20° C. In some embodiments, the storage is conducted at a temperature between 20-30° C. In some embodiments, the storage is conducted at a temperature between 40-50° C. In some embodiments, the storage is conducted at a temperature between 50-60° C. In some embodiments, the storage is conducted for 3-120 months, 3-96 months, 3-72 months, 3-60 months. 3-48 months, 3-36 months, 3-24 months, 3-12 months, 12-120 months, 12-96 months, 12-72 months, 12-60 months, 12-48 months, 12-36 months, 12-24 months, 24-120 months, 24-96 months, 24-72 months, or 24-48 months. In some embodiments, the storage is conducted at 50-100%, 60-100%, 70-100%, 75%-100%, 80-100%, 90-100% or 95-100% relative humidity.

In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 3, 6, 9, 12, 18, 24, 36, 48, 60, or more months of storage above 30° C. (e.g., from about 40° C. to about 50° C.). In some embodiments, the lyophilized compositions comprise mRNA with at least 96% relative purity after 1 month of storage between 30-60° C. In some embodiments, the lyophilized compositions comprise mRNA with at least 93% relative purity after 2 months of storage between 30-60° C. In some embodiments, the lyophilized compositions comprise mRNA with at least 90% relative purity after 3 months of storage between 30-60° C. In some embodiments, the lyophilized compositions comprise mRNA with at least 85% relative purity after 4 months of storage between 30-60° C. In some embodiments, the lyophilized compositions comprise mRNA with at least 85% relative purity after 5 months of storage between 30-60° C. In some embodiments, the lyophilized compositions comprise mRNA with at least 80% relative purity after 6 months of storage between 30-60° C.

In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 6 months of storage at a relative humidity above 50% (e.g., 50-100%, 50-75%, 50-80%, 80-100%, or 60-75%). In some embodiments, the lyophilized compositions comprise mRNA with at least 93% relative purity after 3 months of storage at relative humidity between 70-100%. In some embodiments, the lyophilized compositions comprise mRNA with at least 87% relative purity after 6 months of storage at relative humidity between 70-100%. In some embodiments, the lyophilized compositions comprise mRNA with at least 85% relative purity after 9 months of storage at relative humidity between 70-100%. In some embodiments, the lyophilized compositions comprise mRNA with at least 80% relative purity after 12 months of storage at relative humidity between 70-100%. In some embodiments, the lyophilized compositions comprise mRNA with at least 80% relative purity after 24 months of storage at relative humidity between 70-100%. In some embodiments, the lyophilized compositions comprise mRNA with at least 80% relative purity after 36 months of storage at relative humidity between 70-100%. In some embodiments, the lyophilized compositions comprise mRNA with at least 80% relative purity after 48 months of storage at relative humidity between 70-100%. In some embodiments, the lyophilized compositions comprise mRNA with at least 80% relative purity after 60 months of storage at relative humidity between 70-100%.

In some embodiments, the lyophilized compositions comprise mRNA with at least 90%, at least 91%, at least 92%, at least 93%, at least 94%, at least 96%, at least 97%, at least 98%, or at least 99% relative purity, compared to the purity of mRNA immediately after the conclusion of lyophilization, after 3, 6, 9, 12, 18, 24, 36, 48, 60, or more months of storage between 2-8° C. In some embodiments, the lyophilized compositions comprise mRNA with at least 90% relative purity after 12 months of storage between 2-8° C. In some embodiments, the lyophilized compositions comprise mRNA with at least 90% relative purity after 24 months of storage between 2-8° C. In some embodiments, the lyophilized compositions comprise mRNA with at least 90% relative purity after 36 months of storage between 2-8° C. In some embodiments, the lyophilized compositions comprise mRNA with at least 90% relative purity after 48 months of storage between 2-8° C. In some embodiments, the lyophilized compositions comprise mRNA with at least 90% relative purity after 60 months of storage between 2-8° C. In some embodiments, the lyophilized compositions comprise mRNA with at least 90% relative purity after 72, 96, 108, 120, or more, months of storage between 2-8° C.

In some embodiments, the lyophilized compositions comprise mRNA with at least 80%, at least 85%, at least 90%, at least 91%, at least 92%, at least 93%, at least 94%, at least 96%, at least 97%, at least 98%, or at least 99% relative purity, compared to the purity of mRNA immediately after the conclusion of lyophilization, after 3, 6, 9, 12, 18, 24, 36, 48, 60, or more months of storage between 20-30° C. In some embodiments, the lyophilized compositions comprise mRNA with at least 80% relative purity after 12 months of storage between 20-30° C. In some embodiments, the lyophilized compositions comprise mRNA with at least 80% relative purity after 24 months of storage between 20-30° C. In some embodiments, the lyophilized compositions comprise mRNA with at least 80% relative purity after 36 months of storage between 20-30° C. In some embodiments, the lyophilized compositions comprise mRNA with at least 80% relative purity after 48 months of storage between 20-30° C. In some embodiments, the lyophilized compositions comprise mRNA with at least 80% relative purity after 60 months of storage between 20-30° C. In some embodiments, the lyophilized compositions comprise mRNA with at least 80% relative purity after 72, 96, 108, 120, or more, months of storage between 20-30° C.

In some embodiments, lipid nanoparticles of the lyophilized compositions comprise lipid nanoparticles comprising mRNA with an encapsulation efficiency of 70%. As used herein, “encapsulation efficiency” refers to the percentage of nucleic acid (e.g., mRNA) in a composition that is comprised within lipid nanoparticles. mRNA encapsulated within a lipid nanoparticle is not exposed to the environment outside the lipid nanoparticle, and is thus protected from the action of environmental factors such as nucleases, which can cleave free mRNA. Encapsulation efficiency can be measured by any one of multiple methods known in the art, such as a RiboGreen assay. RiboGreen is a fluorescent dye that is fluorescent only when bound to a nucleic acid. In a RiboGreen assay, one sample of a composition containing mRNA in lipid nanoparticles is subjected to a treatment that disrupts lipid nanoparticle integrity to release encapsulated mRNA, such as exposure to a detergent, while another sample is not disrupted. RiboGreen is then added to both samples, and the fluorescence emitted from both samples is measured. Encapsulation efficiency (E.E.) is calculated by the equation

$$E . E . = ( 1 - \frac{Fluorescence ( non - disrupted sample )}{Fluorescence ( disrupted sample )} ) * 100 \% .$$

In some embodiments, the encapsulation efficiency is at least 70%, at least 75%, at least 80%, at least 85%, at least 90%, at least 91%, at least 92%, at least 93%, at least 94%, at least 95%, at least 96%, at least 97%, at least 98%, at least 99%, or up to 100%.

In some embodiments, RNA encapsulation decreases by about 10% or less, such as about 9% or less, about 8% or less, about 7% or less, about 6% or less, about 5% or less, about 4% or less, about 3% or less, about 2% or less, or about 1% or less immediately after lyophilization and/or after storage following lyophilization. In some embodiments, the amount of encapsulated RNA does not substantially decrease during lyophilization and/or storage of a lyophilized LNP. In some embodiments, the lyophilized compositions comprise mRNA with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, up to 100%, or greater than 100% in vitro potency, relative to the in vitro potency of the mRNA that was present in the composition prior to lyophilization. As used herein, “in vitro potency” of an mRNA refers to the capacity of an mRNA to be translated into a protein encoded by the mRNA during an in vitro translation reaction. In in vitro translation, an mRNA encoding a protein is incubated in the presence of ribosomes and aminoacyl-tRNAs, which allow the encoded protein to be produced in the absence of cells. The in vitro potency of a first mRNA relative to a second mRNA encoding the same protein may be calculated by comparing the amount of protein produced by each mRNA in separate in vitro translation reactions, using the equation

$$\% in vitro potency = \frac{protein produced by first mRNA ( g )}{protein produced by second mRNA ( g )} .$$

Some embodiments comprise reconstituting a lyophilized lipid nanoparticle composition, e.g., in a reconstitution buffer suitable for pharmaceutical administration. In some embodiments, the lyophilized lipid nanoparticle composition is reconstituted in water. In some embodiments, the lyophilized lipid nanoparticle composition is reconstituted in a salt solution (e.g., a sodium chloride solution), such as an about 0.5%, about 0.9%, about 1%, about 1.5%, about 2%, about 5%, about 10%, about 30%, about 40%, about 50%, about 60%, about 70%, about 80%, or about 90% salt solution. In some embodiments, the reconstitution buffer is at a physiological pH (e.g, pH 7-7.5, such as 7.4). Some embodiments comprise a reconstituted lipid nanoparticle composition. In some embodiments, the lyophilized composition is reconstituted (e.g., via agitation, swirling, shaking, and/or repeated pipette aspirating and dispensing) at a temperature of from about 1° C. to about 75° C., such as about 4° C., about 5° C., about 10° C., about 15° C., about 20° C., about 25° C., about 30° C., about 35° C., about 40° C., about 45° C., about 50° C., about 55° C., about 60° C., about 65° C., about 70° C., or about 75° C.

### EXAMPLES

### Example 1: Effect of Sucrose Concentration on Lipid Nanoparticle Size

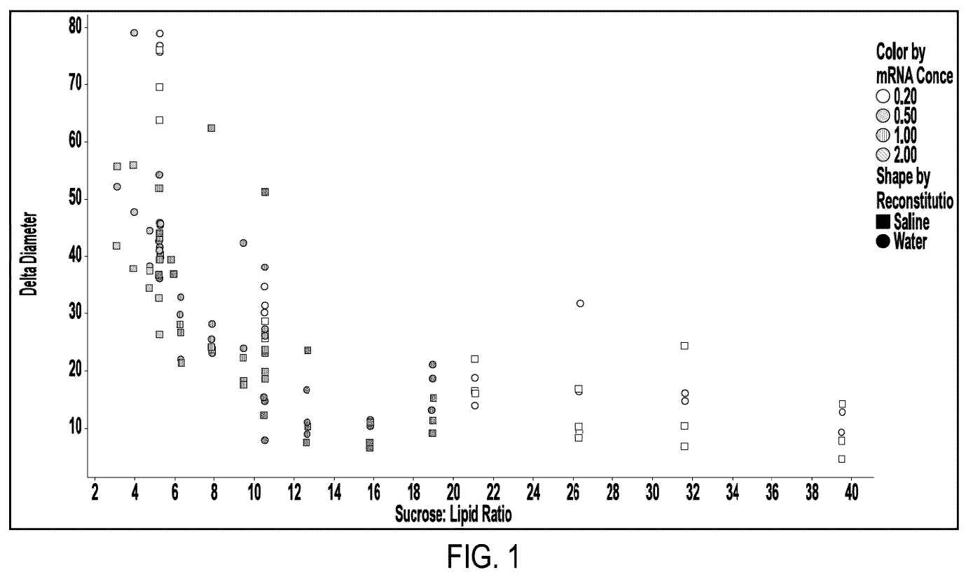

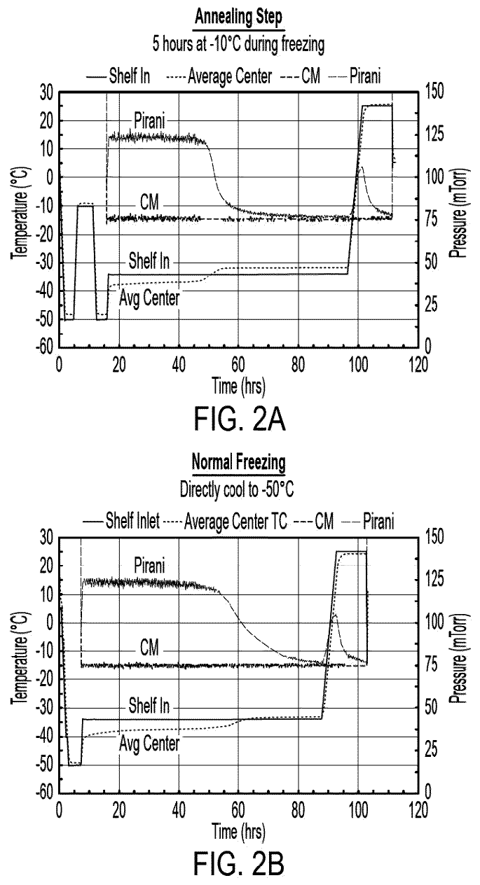

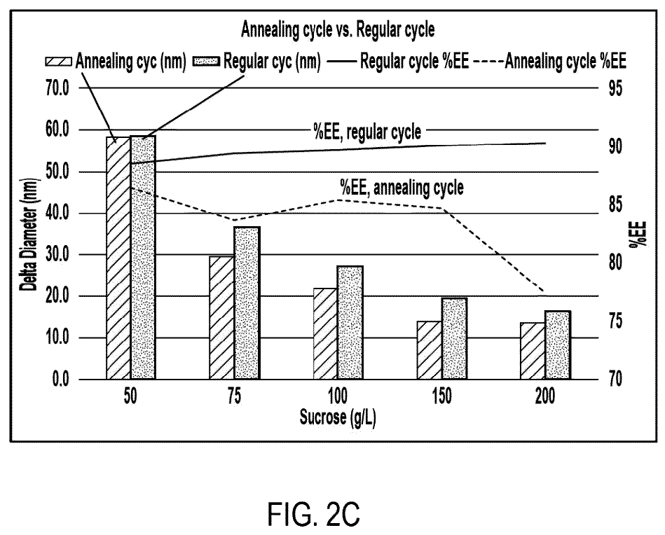

Lipid nanoparticles were produced by combining an aqueous solution comprising mRNA and sucrose with an organic solvent comprising lipids, resulting in the formation of LNPs containing mRNA. Aqueous solutions contained varying amounts of sucrose, such that the ratio of sucrose molecules to lipid molecules in the LNP-mRNA compositions varied from 3:1 to 40:1 (FIG. 1). The size of LNPs produced at each sucrose:lipid ratio were measured, and surprisingly, higher sucrose:lipid ratios resulted in the formation of smaller LNPs, as measured by delta diameter (FIGS. 1 and 2C).

### Example 2: Effect of Lyophilization Process on Stability of Lyophilized Composition

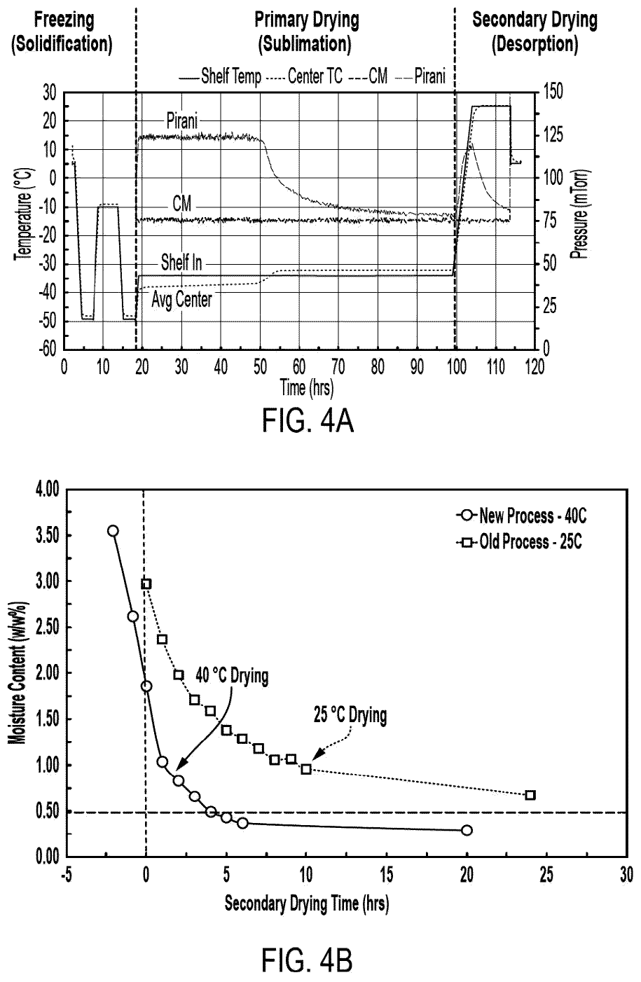

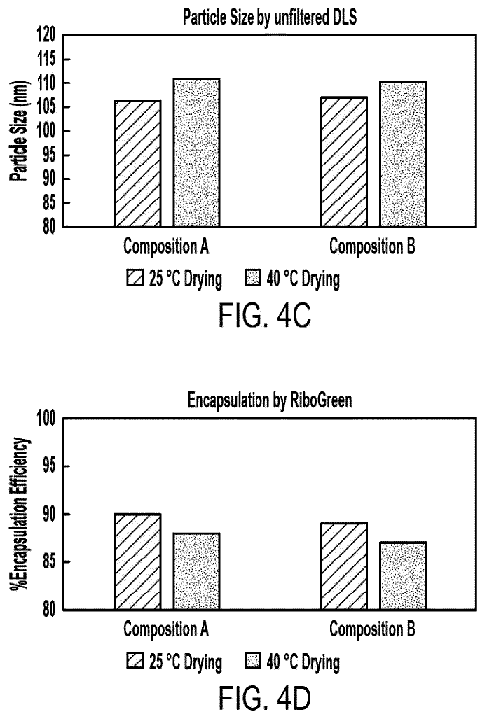

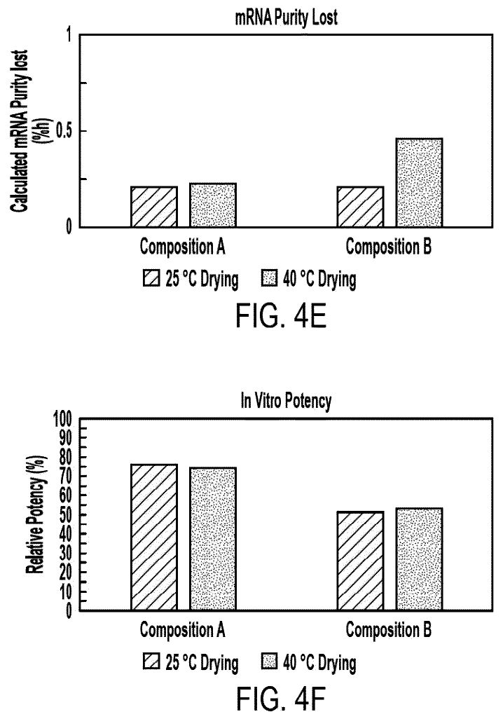

Lyophilization was used to remove moisture from LNP-mRNA compositions produced by the methods described in Example 1. The lyophilization process consisted of three phases: 1) freezing (solidification) phase, 2) primary drying (sublimation) phase, and 3) secondary drying (desorption) phase, shown in FIG. 4A. In the freezing phase, water present in the composition was solidified by exposing the composition to a cold temperature, −50° C. The freezing step consisted of either cooling the compositions directly to −50° C., or holding them at −10° C. for several hours prior to cooling to −50° C. In the primary drying phase, the compositions were exposed to a slightly higher temperature, −35° C., and low pressure (75 mTorr), which allowed for sublimation of a majority of the water in the compositions. The primary drying phase was determined to be complete when the reading of the Pirani gauge was similar to the reading of the capacitance manometer (CM), which is commonly used in the art to evaluate the completion of primary drying. In the secondary drying phase, the compositions were heated to a higher temperature, either 25° C. or 40° C., to allow evaporation of remaining water.

### Annealing Step

During the freezing phase, LNP-mRNA compositions were either briefly cooled to −50° C., then incubated at −10° C. for 5 hours for annealing, and finally incubated at −50° C. until solidification was complete (“Annealing Step”, FIG. 2A), or directly cooled to −50° C. and incubated for approximately 5 hours (“Normal Freezing”, FIG. 2B), As shown in FIG. 2C, the incorporation of an annealing step into the freezing phase resulted in decreased LNP size relative to a freezing phase that did not have an annealing step. This effect was observed across multiple concentrations of sucrose.

### Secondary Drying Phase

Following completion of the primary drying phase, as determined by the Pirani profile, residual water was removed from the composition by a secondary drying phase. An overview of the parameters of the lyophilization process, including the secondary drying phase, is given by FIG. 4A. To test the effect of temperature in the secondary drying phase, compositions were subjected to two distinct lyophilization processes: one in which secondary drying was conducted at 25° C., and one in which secondary drying was conducted at 40° C. As shown in FIG. 4B, secondary drying at a higher temperature resulted in the production of lyophilized LNP-mRNA compositions with moisture contents below 0.5% w/w, which was not achieved by secondary drying at 25° C. This reduction in moisture content was achieved without substantially compromising the integrity of the mRNA, as the 40° C. secondary drying phase only marginally affected the particle size, encapsulation efficiency, mRNA purity, and in vitro potency of lyophilized LNP-mRNA compositions (FIGS. 4C-4F). In each pair of bars in FIGS. 4C-4F, the left bar represents 25° C. drying and the right bar represents 40° C. drying.

### Stability of Lyophilized Compositions

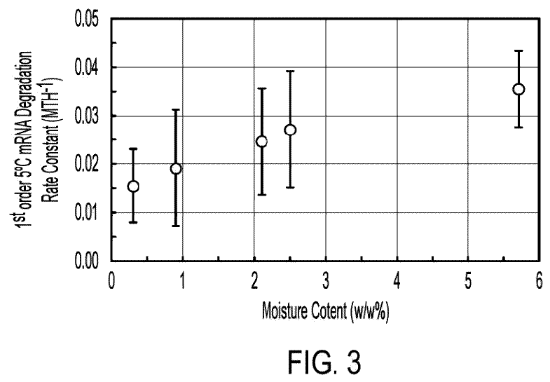

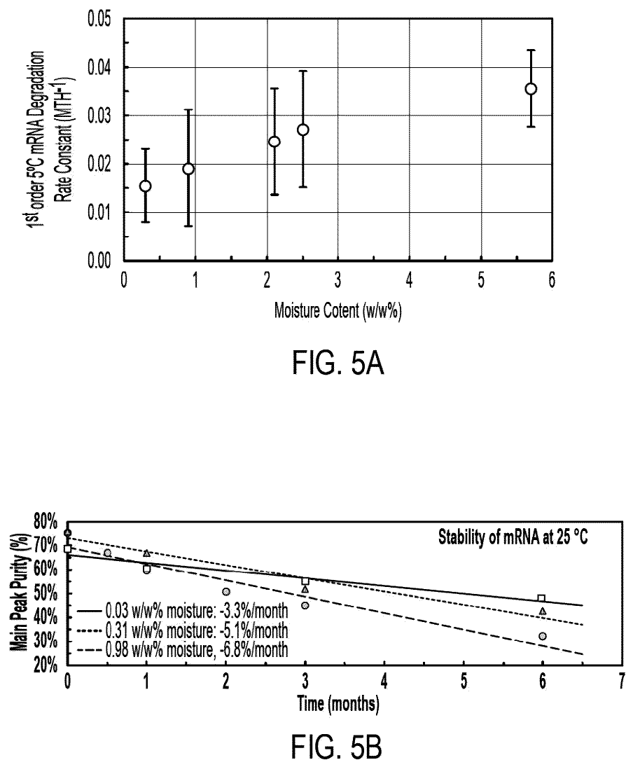

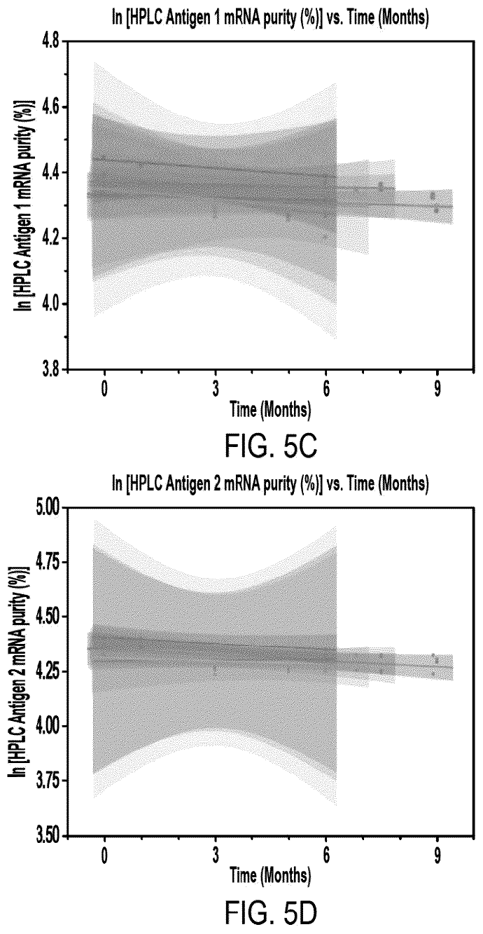

The effect of moisture content on the stability of mRNA in lyophilized LNP-mRNA compositions was evaluated by measuring the mRNA purity over time in compositions with varying moisture contents. From these measurements, the rate constant of mRNA degradation during storage of the LNP-mRNA compositions at 5° C. was regressed (FIGS. 3 and 5A). This analysis revealed that mRNA degrades faster in compositions with higher moisture contents. The purity of mRNA in lyophilized compositions was also measured during storage at 25° C. for several months (FIG. 5B). Compositions with lower moisture content had had reduced initial mRNA purity, but the mRNA in these compositions with lower moisture content also degraded more slowly, and after 6 months of storage, the compositions with the lowest moisture content had more intact, pure mRNA than compositions with higher moisture contents. Thus, the use of a higher temperature in the secondary drying phase resulted in the production of markedly more stable LNP-mRNA compositions, offsetting the slight reduction in initial mRNA purity resulting from high secondary drying temperatures. From these analyses of mRNA stability based on moisture content, the shelf life of lyophilized LNP-mRNA compositions with various moisture contents during storage at 2-8° C. was estimated (Table 1).

TABLE 1
Shelf life of lyophilized LNP-mRNA compositions at 2-8° C.

| Moisture content (% w/w) | Predicted shelf life at 2-8° C. (months) |
|---|---|
| 5.7 | 9.5 ± 1.7 |
| 2.5 | 12.4 ± 3.8 |
| 2.1 | 13.6 ± 4.2 |
| 0.9 | 17.4 ± 6.7 |
| 0.3 | 21.6 ± 7.1 |

In a second experiment, multiple lots of lyophilized LNP-mRNA compositions were stored for several months at 5° C., and the purity of mRNAs in the compositions was monitored over 6 or 9 months (FIGS. 5C-5D). Each lot included an LNP-mRNA composition containing an mRNA encoding a first protein (Antigen 1), and another LNP-mRNA composition containing an mRNA encoding a second protein (Antigen 2). Lyophilized compositions contained moisture contents of 0.20-0.65% w/w. Observations of mRNA purity over 6 or 9 months of storage were used to estimate the first-order degradation constants for each mRNA in lyophilized LNP-mRNA compositions, shown below in Table 2. Based on a starting mRNA purity of 80%, the predicted shelf life of mRNAs (duration of time during which mRNA in lyophilized LNP-mRNA compositions remains at least 50% pure) was also estimated.

TABLE 2
First-order degradation rate estimates for
lyophilized LNP-mRNA compositions at 5° C.

| | | Predicted shelf |
|---|---|---|
| mRNA (encoded | mRNA decay constant at 5° C. | life at 5° C. |
| protein) | (In % purity month⁻¹) | (months) |
| Antigen 1 | −0.0039 ± 0.0019 | 120.5 |
| Antigen 2 | −0.0075 ± 0.0019 | 62.7 |

### Example 3: In Vitro Expression of Proteins from Lyophilized Compositions

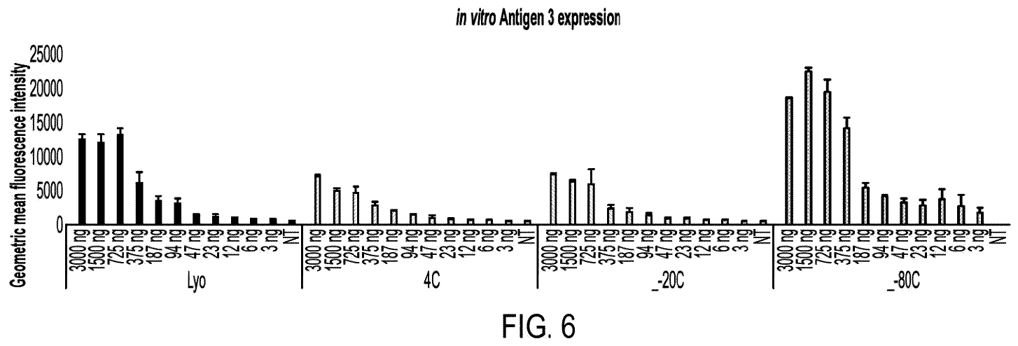

Lipid nanoparticles containing mRNA encoding a protein (Antigen 3) were either 1) lyophilized and reconstituted; 2) stored at 4° C.; 3) frozen at −20° C. and thawed, or 4) frozen at −80° C. and thawed. Compositions were diluted to prepare a series of compositions containing varying amounts of mRNA, and then added to Hep3B cells in 24-well plates to transfect cells with mRNA. After transfection, cells were incubated for 18-20 hours to allow for mRNA translation and Antigen 3 expression. 18-20 hours post-transfection, cells were harvested, stained with an antibody specific to Antigen 3, and the intensity of Antigen 3 expression was measured by flow cytometry. (FIG. 6). mRNA in lyophilized and reconstituted compositions was efficiently expressed after delivery to cells, with lyophilized compositions resulting in greater Antigen 3 production than compositions stored at 4° C. or −20° C.

### Example 4: Immunization with Lyophilized Compositions

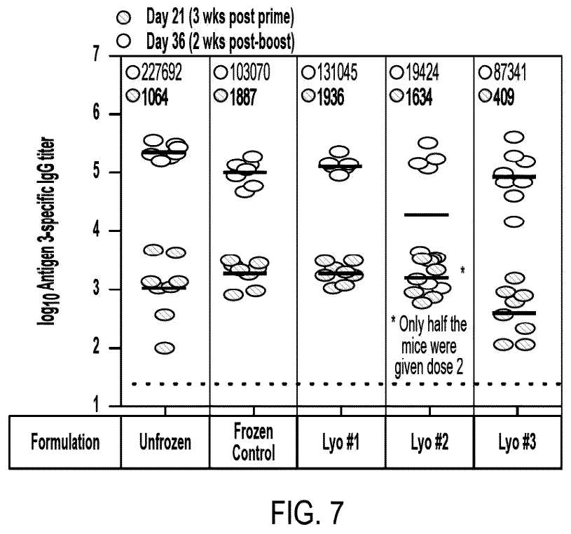

Three distinct formulations of lipid nanoparticles, each containing an mRNA encoding a protein antigen (Antigen 3) were lyophilized and reconstituted to prepare LNP-mRNA vaccine compositions (Lyo #1, Lyo #2, Lyo #3). Additionally, a composition containing the same mRNA was frozen and thawed (Frozen Control). Finally, a composition containing the same mRNA was prepared without freezing or lyophilization (Unfrozen). Female BALB/c, 6-8 weeks of age, were administered a dose of one of the prepared compositions containing 3 μg of mRNA by intramuscular injection (prime dose, day 0). Three weeks later, mice were administered another 3 μg dose of the same composition (boost dose, day 22). Sera were collected from mice at day 21 (3 weeks post-prime, before boost) and day 36 (2 weeks post-boost), and antibodies specific to Antigen 3 in the serum of each mouse were quantified by ELISA (FIG. 7). For each group, the geometric mean antibody titers after the prime dose (bottom number) and boost dose (top number) are shown above the data points. In the Lyo #2 group, only half the mice received a boost dose, and so some data points reflect antibody titers at day 36, five weeks after the first dose.

Lyophilized compositions generated Antigen 3-specific antibodies at titers that were approximately equivalent to those generated by frozen compositions (FIG. 7). These results indicate that mRNA in lyophilized compositions is capable of being translated both in vitro and in vivo, with translated proteins being able to exert a therapeutic effect, such as inducing the production of protein-specific antibodies after administration to a subject. Compositions containing lipid nanoparticles and mRNA can thus be lyophilized for prolonged stability and ease of transport, with mRNA retaining the ability to be translated following reconstitution and delivery to cells.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in some embodiments, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in some embodiments, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. Each possibility represents a separate embodiment of the present invention.

It should be understood that, unless clearly indicated to the contrary, the disclosure of numerical values and ranges of numerical values in the specification includes both i) the exact value(s) or range specified, and ii) values that are “about” the value(s) or ranges specified (e.g., values or ranges falling within a reasonable range (e.g., about 10% similar)) as would be understood by a person of ordinary skill in the art.

It should also be understood that, unless clearly indicated to the contrary, in any methods disclosed herein that include more than one step or act, the order of the steps or acts of the method is not necessarily limited to the order in which the steps or acts of the method are disclosed.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of preparing a lyophilized composition, the method comprising lyophilizing a composition that comprises a lipid nanoparticle encapsulating an mRNA.

2. The method of claim 1, wherein the composition comprises a lyoprotectant.

3. The method of claim 2, wherein the lyoprotectant comprises a sugar.

4. The method of claim 2 or 3, wherein the lyoprotectant comprises sucrose.

5. The method of any one of claims 2-4, wherein the lipid nanoparticle comprises one or more lipids, and where the lipid nanoparticle composition comprises a lyoprotectant mass:lipid mass ratio of at least 5:1.

6. The method of claim 5, wherein the lyoprotectant mass: lipid mass ratio is about 6:1 to about 40:1, optionally wherein the lyoprotectant mass:lipid mass ratio is about 6:1, 7:1, 8:1, 9:1, 10:1, 15:1, 20:1, 20:1, 30:1, or 40:1.

7. The method of any one of claims 1-6, wherein the composition comprises a buffer.

8. The method of claim 7, wherein the buffer is selected from the group consisting of a Tris buffer, citrate buffer, phosphate buffer, triethylammonium bicarbonate (TEAB), and histidine buffer.

9. The method of claim 8, wherein the buffer is a Tris buffer.

10. The method of any one of claims 7-9, wherein the concentration of the buffer in the composition is about 1 mM to about 100 mM.

11. The method of claim 10, wherein the concentration of the buffer is about 10 mM to about 30 mM.

12. The method of any one of claims 1-11, wherein the composition has a pH of about 6.5 to about 8.5.

13. The method of claim 12, wherein the composition has a pH of about 7 to about 8.

14. The method of claim 13, wherein the composition has a pH of about 7.4 to about 8.

15. The method of any one of claims 1-14, wherein the lyophilizing comprises an annealing step.

16. The method of claim 15, wherein the annealing step comprises exposing the lipid nanoparticle composition to a first temperature above the freezing temperature of the composition and a second temperature below the freezing temperature of the composition.

17. The method of claim 16, wherein the first temperature is from about −30° C. to about 0° C.

18. The method of claim 17, wherein the first temperature is about −30° C., about −25° C., about −20° C., about −15° C., about −10° C., about −5° C., or about 0° C.

19. The method of any one of claims 16-18, wherein the second temperature is from about −100° C. to about −30° C.

20. The method of claim 19, wherein the second temperature is about −100° C., about −90° C., about −80° C., about −70° C., about −60° C., about −50° C., about −40° C., or about −30° C.

21. The method of any one of claims 16-20, further comprising exposing the lipid nanoparticle composition to a third temperature before exposing the lipid nanoparticle composition to the first temperature, wherein the third temperature is from about −100° C. to about −30° C.

22. The method of claim 21, wherein the third temperature is about −100° C., about −90° C., about −80° C., about −70° C., about −60° C., about −50° C., about −40° C., or about −30° C.

23. The method of any one of claims 15-22, wherein the annealing step is conducted for at least 2 hours, at least 3 hours, at least 4 hours, at least 5 hours, at least 10 hours, or at least 15 hours.

24. The method of any one of claims 16-23, wherein the lipid nanoparticle composition is exposed to the first temperature for a first period of from about 2 to about 6 hours, the second temperature for a second period of from about 2 to about 6 hours, and/or the third temperature for a third period of from about 2 to about 6 hours.

25. The method of any one of claims 1-24, wherein the lyophilizing comprises a sublimation step.

26. The method of claim 25, wherein the sublimation is performed at a vacuum pressure of from about 50 mTorr and about 300 mTorr.

27. The method of any one of claims 1-26, wherein the lyophilizing comprises a desorption step, wherein the desorption step comprises exposing the composition to a desorption temperature.

28. The method of claim 27, wherein the desorption temperature is about 10° C. or higher.

29. The method of claim 28, wherein the desorption temperature is about 10° C., about 15° C., about 20° C., about 25° C., about 30° C., about 35° C., about 40° C., about 45° C., about 50° C., about 55° C., or about 60° C.

30. The method of any one of claims 27-29, wherein the desorption step comprises exposing a sublimated composition to a desorption temperature of about 40° C.

31. The method of any one of claims 27-30, wherein the desorption is conducted for at least 2 hours.

32. The method of claim 31, wherein the desorption is conducted for about 5 hours, about 10 hours, about 15 hours, or about 20 hours.

33. The method of any one of claims 27-32, wherein the desorption is performed at a vacuum pressure of from about 50 mTorr and about 300 mTorr.

34. The method of any one of claims 1-33, wherein the lyophilized composition has a moisture content of 6.0% w/w or less 5.0% w/w or less, 4.0% w/w or less, 3.5% w/w or less, 3.0% w/w or less, 2.5% w/w or less, 2.0% w/w or less, 1% or less, 0.5% or less, 0.3% or less, or 0.25% or less.

35. The method of any one of claims 1-34, wherein a coefficient of degradation at 5° C. of the nucleic acid in the lyophilized composition is 0.05 month⁻¹ or less, 0.04 month⁻¹ or less, 0.03 month⁻¹ or less, or 0.02 month⁻¹ or less.

36. The method of claim 35, wherein the coefficient of degradation is 0.01 month⁻¹ or less, 0.008 month⁻¹ or less, 0.006 month⁻¹ or less, or 0.004 month⁻¹ or less.

37. The method of any one of claims 1-36, wherein the mRNA in the lyophilized composition is at least 50% pure after least 12 months, at least 18 months, at least 21 months, at least 24 months, at least 27 months, at least 30 months, at least 33 months, or at least 36 months of storage.

38. The method of claim 37, wherein the storage is conducted at a temperature between about 2° C. and about 8° C.

39. The method of claim 38, wherein the storage is conducted at about 5° C.

40. The method of any one of claims 37-39, wherein the mRNA in the lyophilized composition is at least 50% pure after at least 48 months or at least 60 months of storage at about 5° C.

41. The method of any one of claims 1-40, wherein the lipid nanoparticle comprises: an ionizable amino lipid.

42. The method of claim 41, wherein the lipid nanoparticle further comprises: a non-cationic lipid; a sterol; and a polyethylene glycol (PEG)-modified lipid.

43. The method of claim 42, wherein the lipid nanoparticle comprises: 40-55 mol % ionizable amino lipid; 5-15 mol % non-cationic lipid; 35-45 mol % sterol; and 1-5 mol % PEG-modified lipid.

44. A lyophilized composition produced by a method of any one of claims 1-43.

45. A lyophilized pharmaceutical composition comprising a lipid nanoparticle encapsulating an mRNA.

46. The lyophilized pharmaceutical composition of claim 45, wherein the lyophilized pharmaceutical composition comprises a lyoprotectant.

47. The lyophilized pharmaceutical composition of claim 46, wherein the lyoprotectant comprises a sugar.

48. The lyophilized pharmaceutical composition of claim 46 or 47, wherein the lyoprotectant comprises sucrose.

49. The lyophilized pharmaceutical composition of any one of claims 46-48, wherein the lipid nanoparticle comprises one or more lipids, and where the lyophilized pharmaceutical composition comprises a lyoprotectant mass:lipid mass ratio of at least 5:1.

50. The lyophilized pharmaceutical composition of claim 49, wherein the lyoprotectant mass:lipid mass ratio is about 6:1 to about 40:1, optionally wherein the lyoprotectant mass:lipid mass ratio is about 6:1, 7:1, 8:1, 9:1, 10:1, 15:1, 20:1, 20:1, 30:1, or 40:1.

51. The lyophilized pharmaceutical composition of any one of claims 45-50, wherein the lyophilized composition has a moisture content of 6.0% w/w or less, 5.0% w/w or less, 4.0% w/w or less, 3.5% w/w or less, 3.0% w/w or less, 2.5% w/w or less, or 2.0% w/w or less, 1% or less, 0.5% or less, 0.3% or less, or 0.25% or less.

52. The lyophilized pharmaceutical composition of any one of claims 45-51, wherein a coefficient of degradation at 5° C. of the nucleic acid in the lyophilized composition is 0.05 month⁻¹ or less, 0.04 month⁻¹ or less, 0.03 month⁻¹ or less, or 0.02 month⁻¹ or less.

53. The lyophilized pharmaceutical composition of claim 52, wherein the coefficient of degradation is 0.01 month⁻¹ or less, 0.008 month⁻¹ or less, 0.006 month⁻¹ or less, or 0.004 month⁻¹ or less.

54. The lyophilized pharmaceutical composition of any one of claims 45-53, wherein the mRNA in the lyophilized composition is at least 50% pure after least 12 months, at least 18 months, at least 21 months, at least 24 months, at least 27 months, at least 30 months, at least 33 months, or at least 36 months of storage.

55. The lyophilized pharmaceutical composition of claim 54, wherein the storage is conducted at a temperature between about 2° C. and about 8° C.

56. The lyophilized pharmaceutical composition of claim 55, wherein the storage is conducted at about 5° C.

57. The lyophilized pharmaceutical composition of any one of claims 54-56, wherein the mRNA in the lyophilized composition is at least 50% pure after at least 48 months or at least 60 months of storage at about 5° C.

58. The lyophilized pharmaceutical composition of any one of claims 45-57, wherein the lipid nanoparticle comprises: an ionizable amino lipid.

59. The lyophilized pharmaceutical composition of any one of claims 45-58, wherein the lipid nanoparticle further comprises: a non-cationic lipid; a sterol; and a polyethylene glycol (PEG)-modified lipid.

60. The lyophilized pharmaceutical composition of claim 59, wherein the lipid nanoparticle comprises: 40-55 mol % ionizable amino lipid; 5-15 mol % non-cationic lipid; 35-45 mol % sterol; and 1-5 mol % PEG-modified lipid.

61. A method comprising reconstituting the lyophilized pharmaceutical composition of any one of claims 44-60.
